(19) United States
(12) Patent Application Publication
(10) Pub. No.: US 2008/0318893 A1
(43) Pub. Date: Dec. 25, 2008

(54) Blockade of Calcium Channels

(75) Inventor: Eduardo Marban, Beverly Hills, CA (US)

Correspondence Address: BANNER & WITCOFF, LTD., 1100 13th STREET, N.W., SUITE 1200, WASHINGTON, DC, 20005-4051, US

(73) Assignee: THE JOHNS HOPKINS UNIVERSITY, BALTIMORE, MD (US)

(21) Appl. No.: 12/091,653

(22) PCT Filed: Oct. 26, 2006
(86) PCT No.: PCT/US2006/041696

§ 371 Date: Sep. 5, 2008

Related U.S. Application Data
(60) Provisional application No. 60/730,754, filed on Oct. 27, 2005.

(51) Int. Cl. A61K 31/7105; A61P 9/04

(52) U.S. Cl. 514/44

(57) ABSTRACT

Knock-down of L-type calcium channel beta subunit (LTCCβ) attenuates the hypertrophic response both in vitro and in vivo without compromising systolic performance. Knock-down can be accomplished by administration of a vector encoding a short hairpin RNA which specifically modulates expression of LTCCβ. Suppression of the LTCCβ expression represents a therapeutic modality for cardiac hypertrophy.

This application claims the benefit of provisional application U.S. Ser. No. 60/730,754 filed 27 Oct. 2005, the disclosure of which is expressly incorporated herein.

### TECHNICAL FIELD OF THE INVENTION

This invention is related to the area of L-type calcium channels. In particular, it relates to disease states related to excessive calcium channel activity.

### BACKGROUND OF THE INVENTION

The development of LVH, irrespective of etiology, confers an incremental risk of adverse outcomes in the general population and in patients with different forms of cardiovascular disease¹. Importantly, LVH is also an early event in patients destined to develop congestive heart failure^{1,2}. Mechanistically, enhancement of calcium-regulated signaling pathways underlies the development of LVH³. Calcium cycling in the heart is triggered by calcium influx through L-type calcium channels⁴. Thus, calcium channel blockade is a logical therapeutic approach. L-type calcium channels are present and functionally important not only in cardiac myocytes but also in diverse smooth muscles and in neurons. While pharmacological blockade of the L-type calcium channels has proven to reduce left ventricular mass in hypertensive subjects, improvement on cardiovascular mortality has not been demonstrated with these agents⁵. Undesired effects due to blockade of non-cardiac channels could account in part for the limited clinical benefit observed with these agents.

L-type calcium channels are heteromultimers of various subunits. Work in heterologous expression systems indicates that the accessory β subunit (LTCCβ) not only favors the trafficking of the calcium channel to the surface membrane^{6,7}, but also enhances the probability of channel opening^{8,9} resulting in increased calcium current. Interestingly, far less is known about the role of LTCCβ in native cardiac cells.

There is a continuing need in the art to treat disease states caused by excessive calcium channel activity.

### SUMMARY OF THE INVENTION

According to a first embodiment a method is provided for treating cardiac hypertrophy in a mammal. A vector encoding a short hairpin RNA is administered to the mammal. The RNA comprises a segment in its double stranded portion that is complementary to an L-type calcium channel accessory β-subunit (LTCCβ) coding sequence. The short hairpin RNA is expressed in the mammal causing one or more physiological effects. Possible physiological effects include:
a. reduced peak calcium current density;
b. reduced calcium current amplitude;
c. suppression of phenylephrine-stimulated protein synthesis;
d. suppression of phenylephrine-stimulated cell size increase;
e. decreased cardiac ventricular wall thickness; and
f. attenuated hypertrophy.

According to another embodiment a method is provided for decreasing calcium channel activity in a mammal. A vector encoding a short hairpin RNA is administered to the mammal. The RNA comprises a segment in its double stranded portion that is complementary to an L-type calcium channel accessory β-subunit (LTCCβ) coding sequence. The short hairpin RNA is expressed in the mammal causing one or more physiological effects. Possible physiological effects include:
a. reduced peak calcium current density;
b. reduced calcium current amplitude;
c. suppression of phenylephrine-stimulated protein synthesis;
d. suppression of phenylephrine-stimulated cell size increase;
e. decreased cardiac ventricular wall thickness; and
f. attenuated hypertrophy.

These and other embodiments which will be apparent to those of skill in the art upon reading the specification provide the art with additional methods for treating disorders of electrically excitable tissues in the body.

### BRIEF DESCRIPTION OF THE DRAWINGS

FIG. 1A-1E. Suppressed ICaL in LTCCB siRNA Transfected NRCMs. FIG. 1A). Schematic diagram of the LTCCB gene and target DNA sequence of the rhodamine-tagged siRNA. FIG. 1B). NRCMs 48 hours after transfection with siRNA duplexes. FIG. 1C). Representative barium current in NRCMs 72 hours after transfection with NS siRNA oligos. And FIG. 1D). B2D2 siRNA oligos. FIG. 1E). Mean current density (pA/pF) voltage relationships in NS (n=3) and B2D2 (n=4) siRNA transfected cells.

FIG. 2A-2C. Vector-based expression of an shRNA attenuates LTCCB gene expression and efficiently transduces NRCMs. FIG. 2A). Schematic diagram of the vector PPT.CG.H1 and shRNA template. FIG. 2B) HEK293 cells (left panels) co-transfected with β2-GFP fusion and either PPT.H1.β2 (lower panel) or PPT.H1.NS (upper panel); FACS quantification of fluorescence reduction is shown on the right (*p<0.05). FIG. 2C. Transmitted light (left) and fluorescence microscopy images (right) of NRCMs transduced at an MOI of 50. GFP fluorescence indicates a transduction efficiency of ≅90%.

FIG. 3A-3C. Reduced calcium transient amplitude in NRCMs transduced with PPT.CG.H1.B2. FIG. 3A). Representative confocal microscopy montage revealing the peak fluorescence emission from field-stimulated NRCMs following loading with the calcium sensitive dye, Rhod-2. FIG. 3B). Representative tracings of Rhod-2 fluorescence intensity as a function of time from cells transduced with PPTCG.H1.NS or PPTCG.H1.B2. FIG. 3C). Mean calcium transient amplitude (F/Fo) fluorescence in PPT.CG.H1.NS (n=103) and PPT.CG.H1.B2 (n=102) cells from three independent experiments. (*p<0.05).

FIG. 4A-4C. Attenuation of PE-induced hypertrophy in NRCMs. FIG. 4A). Representative confocal images of NRCMs 48 hours after PE stimulation revealing significantly reduced cell area in cells transduced with PPT.CG.H1.B2. FIG. 4B). Mean cell area of PPT.CG.H1.NS (n=31) and PPT.CG.H1.B2 (n=19) transduced cells 48 hours after PE stimulation confirms the microscopic findings. FIG. 4C) [H³]leucine incorporation in control, PPT.CG.H1.NS and PPT.CG.H1.B2 (n=9 each) transduced cells with (PE+) and without (PE−) phenylephrine stimulation. (*p<0.05, PE+ compared to PE−).

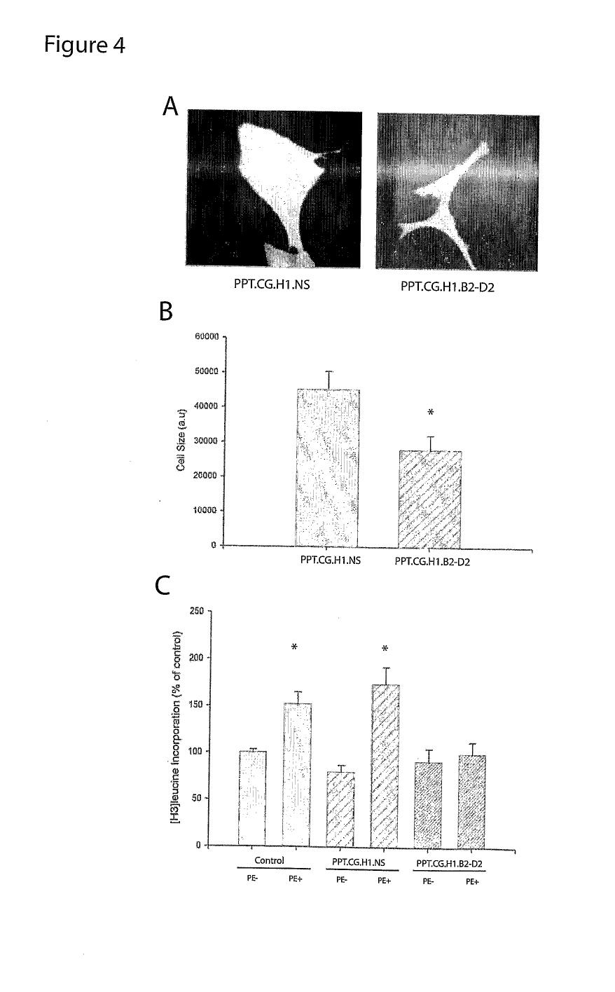

FIG. 5A-5B. In vivo cardiac gene transfer efficiency. Transduction efficiency was assessed four weeks after intracardiac injection of PPT.CMV.LacZnls. FIG. 5A) Representative phase contrast image (40×) showing diffuse expression of nuclear localized β-galactosidase. FIG. 5B) Mean total (n=3) and X-Gal positive nuclei (n=3) per random (20×) high-power field.

FIG. 6A-6C. Attenuation of the hypertrophy response in rats with aortic banding. FIG. 6A) Representative short-axis and m-mode images four weeks after gene transfer and aortic banding showing reduced wall thickness in PPT.CG.H1.B2 injected rats. FIG. 6B) Mean LV wall thickness of PPT.CG.H1.NS (n=7) and PPT.CG.H1.β2 (n=6) injected rats was measured at baseline, 2, and 4 weeks after aortic banding. Attenuation of the hypertrophic response was evident at 2 weeks and persisted to the 4 week time point. FIG. 6C) Heart weight/body weight relationships was also measured in these rats at four weeks after aortic banding. (*p<0.05).

FIG. 7A-7D. No evidence of heart failure in rats with attenuated hypertrophy despite persistence of pressure over-load. FIG. 7A) Body weight, FIG. 7B) heart rate, FIG. 7C) LV diastolic diameter (LVDD) and FIG. 7D) mean LV ejection fraction were comparable four weeks after aortic banding between sham operated (n=4), PPT.CG.H1.NS (n=7) and PPT.CG.H1.β2 (n=6) transduced rats.

### DETAILED DESCRIPTION OF THE INVENTION

It is a discovery of the present inventor that delivery of a short hairpin RNA inhibits expression of LTCC and decreases calcium current activity. This is useful, inter alia, for treating cardiac hypertrophy in mammals. It has been found that such a short hairpin RNA can reduce peak calcium current density; reduce calcium current amplitude; suppress of phenylephrine-stimulated protein synthesis; suppress phenylephrine-stimulated cell size increase; decrease cardiac ventricular wall thickness; and attenuate hypertrophy. The quantitative change that occurs may be at least 5, at least 10, at least 15, at least 20, at least 25, at least 30, at least 35%. These parameters can be measured by standard electrophysiological assays as are known in the art. See Fogoros R N. Electrophysiologic Testing Blackwell Science, Inc. (1999) for general disclosure relating to performing such assays.

The method of the present invention involves delivery to a mammal a vector which encodes the short hairpin RNA. The vector can be any known in the art. Useful properties of the vector include persistence and low pathogenicity. One such vector which can be used is a lentivirus vector. Other viral or non-viral vectors can be used as are known and available in the art. These include plasmids (naked DNA) and liposomes (non-viral) as well as herpes virus, adenovirus and adeno associated virus vectors.

The short hairpin RNA will preferably target the beta subunit of an L-type calcium channel accessory (LTCC) coding sequence (also known as Cacnb2). Other subunits can be targeted including the alpha-1 or alpha2/delta. The targeting is by virtue of sequence complementarity. The complementary sequence is in the stem of the hairpin structure. According to one embodiment, the sequence complementarity is in the conserved D2 region of the coding sequence. Complementary regions may be to any of regions D1-D5. The LTCC coding sequence can be any that is known in the art, including any of the known variant sequences of human LTCC or a rodent sequence such as the Rattus norvegicus or Mus musculus sequence. See SEQ ID NO: 1-9, for example for mammalian coding sequences. One particular complementary sequence is shown in SEQ ID NO: 10.

Diseases which can be treated according to the present invention include but are not limited to cardiac hypertrophies. These may be hypertrophic obstructive cardiomyopathy, left ventricular hypertrophy, hypertrophic cardiomyopathy, right ventricular hypertrophy, hypertensive heart disease, chronic heart failure, asymmetric septal hypertrophy, systemic hypertension and pulmonary hypertension. Any disease associated with increased calcium channel activity can be treated.

Treatment of mammals according to the present invention can be accomplished using just the vector of the invention or a combination of the vector and a drug. The two modes of treatment may be administered concurrently or serially. Such drugs may include calcium channel blockers such as certain phyneylalkylamines, dihydropyridines, benzothiazepines, diphenylpipazines and diarylaminopropylamine. More particular calcium channel blockers include the following: amlodipine (NORVASC™), bepridil (VASCOR™), diltiazem (CARDIZEM™), felodipine (PLENDIL™), isradipine (DYNACIRC™), nicardipine (CARDENE™), nifedipine (ADALAT™), nimodipine (NIMOTOP™), and verapamil (CALAN™). See Roberston, R. M and D. Robertson, supra, (disclosing use of various calcium channel blockers to treat cardiovascular disorders).

The fundamental role of calcium influx through the LTCC in normal excitation-contraction coupling, and the significance of calcium mishandling in heart disease, have recently been reviewed⁴. The importance of the LTCC as a therapeutic target for LVH has been confirmed in many animal models that demonstrate reduction in hypertrophy by calcium channel blockers²². However, there are only limited data on the effect of L-type calcium channel blockade on cardiac hypertrophy beyond blood pressure control^{3,23}. Clinically, calcium channel antagonists have proven to decrease blood pressure and induce regression of LVH, but prolonged survival with these agents has not been demonstrated⁵. Calcium channel blockade of non-cardiac channels explains many of the undesired effects (e.g., systemic hypotension, constipation, edema) of these agents and could also account for the limited effect on cardiovascular mortality. We have previously shown that genetic calcium channel blockade can be achieved in the heart, by over-expressing the small G-protein, Gem, by adenoviral gene transfer²⁴. In the present study, we employ exciting developments in gene regulation and gene transfer technology to achieve persistent LTCC blockade. By incorporating a shRNA expression cassette into an ‘advanced’ generation lentiviral vector we were able to target the LTCCβ in a gene specific manner and achieve long-term modulation of cardiac calcium influx. Gene-silencing of LTCCβ in native cardiac cells suppressed I_{CaL} and decreased calcium transients by 34%, demonstrating the importance of the endogenous levels of LTTCβ for the regulation of calcium handling. This dramatic suppression in I_{CaL} accompanied by a modest reduction in calcium transients, could be explained by the ability of NRCMs to regulate calcium influx by alternative mechanisms such as reverse mode function of the sodium-calcium exchnager^{25,26}. Different levels of LTCCβ gene silencing achieved by siRNA duplexes compared to vector-driven shRNA gene-silencing could also account for this observation. In a cellular model of hypertrophy, down-regulation of LTCCβ expression prevented an increase in relative cell size and abrogated PE-induced protein synthesis. These in vitro findings contribute to the body of evidence of the key role of calcium-regulated signaling pathways in the development of cardiac hypertrophy.³

In pressure-overload conditions, enhanced calcium-regulated signaling also plays a central role in the development of LVH.^{3,27}. While initially considered a “compensatory” response projected to normalize wall stress and facilitate systolic performance²⁸, recent studies have challenged this premise^{27,29}. The inhibition of calcium-regulated signaling pathways, has been shown to abolish pressure overload-induced LVH without compromising systolic function²⁹. In our in vivo study system, modulation of LTCCβ attenuated the development of LVH as demonstrated by a relative decrease in LVWT and HW/BW. The attenuated hypertrophic response in the setting of a “fixed” aortic stenosis support the evidence of the cardiac effect of LTCCβ knock-down independently of any changes on peripheral vascular resistance.

Although a depressed cardiac contractility might be expected from LTCCβ gene silencing, no changes in systolic performance, as assessed by echocardiographic shortening fraction, were detected during follow-up. Moreover, no signs of impaired cardiac performance such as respiratory distress, or fluid retention were detected. Overexpression of LTCCβ by adenoviral gene transfer has recently shown to induce calcium overload and apoptosis in adult feline cardiomyocytes³⁰. Although an anti-apoptic affect of LTCCβ modulation could also explain the preserved shortening fraction in our in vivo model, further studies would need to be performed to confirm this hypothesis.

Post-transcriptional gene silencing by RNA interference represents a novel tool for modulation of specific genes. Knock-down of endogenous expression of LTCCβ may represent a new approach for studying calcium regulated signaling pathways and the development of new therapeutic strategies for diverse cardiac conditions. For example, pharmacological calcium channel blockers are among the first-line treatment for patients with hypertrophic obstructive cardiomyopathy (HOCM)³¹, to reduce outflow tract obstruction. In accordance with this concept, surgical and more recently non-surgical septal reduction techniques have been developed as a means of treating HOCM with severe LV outflow obstruction.³²⁻³⁴ However, the utility of this therapy is limited by its side effects, including inflammation, fibrosis, and arrhythmogenesis. Focal modulation of LTCCβ by a vector capable of chronically suppressing gene expression may represent an attractive alternative in HOCM. Regional modification of endogenous LTCCβ may improve outflow obstruction by reducing septal hypertrophy without impairing global cardiac hemodynamics.

The present strategy, associated with a vector capable of persistently modulating the expression of an accessory subunit of the LTCC in the heart represents a novel and specific research and therapeutic tool for LVH and other cardiac diseases associated with calcium mishandling.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Methods

shRNA Design and Vector Production.

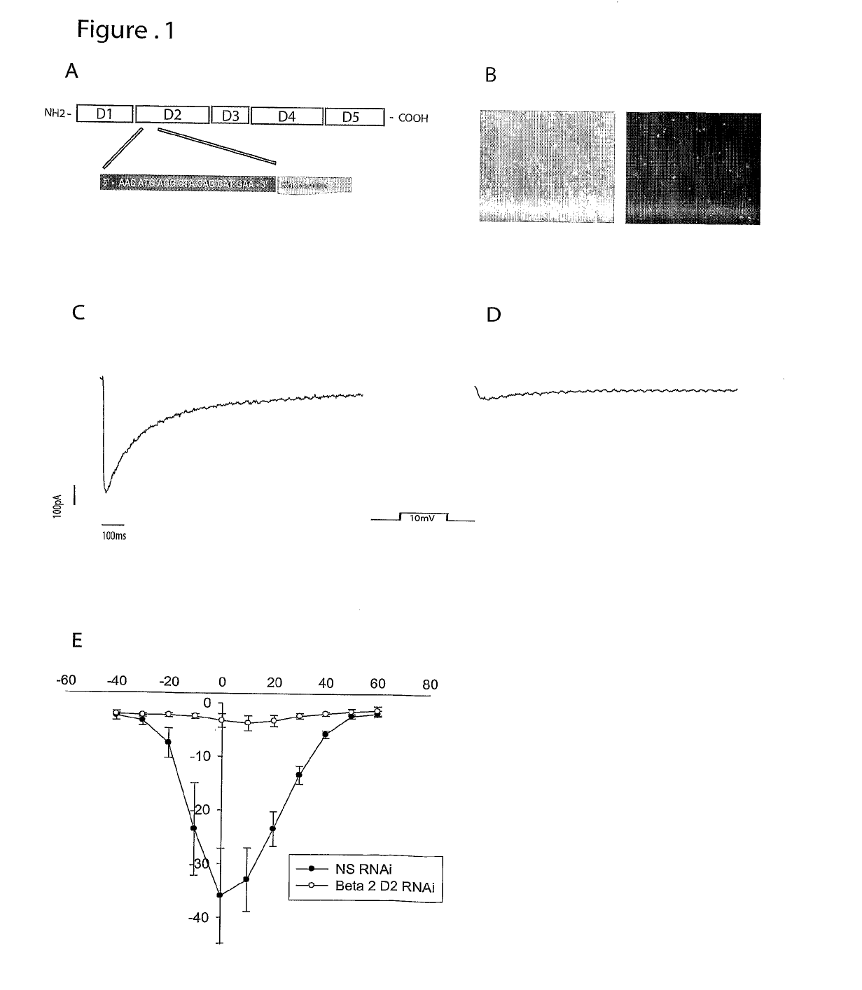

A series of 21 nucleotide short interference RNA duplexes (siRNAs) against the D2 conserved domain¹⁰ of LTCCβ (NM_{—}053851) were designed according to published algorithms¹¹ and synthesized (Qiagen). A total of ten siRNA duplexes were screened by western blot analysis (data not shown). The functional effect of the most suppressive sequence (FIG. 1 A) was confirmed using the whole cell patch clamp technique. This sequence and one scrambled, non-silencing (NS) sequence were designed into a short-hairpin RNA (shRNA) oligonucleotide template consisting of sense, hairpin loop, antisense and terminator sequences all of which were flanked by restriction enzyme sites to facilitate directional subcloning. These oligonucleotides were subcloned into an shRNA expression cassette composed of an RNA polymerase III promoter (H1). The entire shRNA expression cassette was incorporated into the KpnI site of lentiviral vector plasmid pRRLsin18.cPPT.CMV.eGFP.Wpre (provided by Dr. Inder Verma, Salk Institute) (FIG. 1A). The resulting vectors encoded eGFP under the transcriptional control of a CMV promoter and either shRNA against LTCCβ (PPT.CGH1.β2) or a non-silencing shRNA (PPT.CGH1.NS) under the control of the H1 promoter. For the heterologous co-transfection experiments (see below) CMV-GFP was removed resulting in shRNA vectors PPT.H1.β2 and PPT.H1.NS. For viral vector production from these plasmids, the four-plasmid transient transfection of 293 cells was performed as previously described¹²⁻¹⁴. Briefly, vector containing supernatant was collected 48-72 hours after transfection, 0.2 um filter-purified and concentrated by ultracentrifugation (50,000 g for 120 minutes at 10° C.). The viral pellet was resuspended in PBS. Transduction unit (TU) titre was assessed on HEK293 cells in the presence of Polybrene 8 μg/mL (Sigma-Aldrich). Titers of 2-5×10⁸ TU/ml were routinely achieved. For in vivo gene transfer efficiency experiments, the cDNA for nuclear-localized β-galactosidase (LacZnls) was subcloned in place of the GFP gene and vector produced as described.

### Co-Transfection Heterologous Expression.

The LTCCβ-GFP fusion gene was subcloned into an expression plasmid designated pLTCCβ-GFP. In order to establish the knockdown efficacy of PPT.CG.H1.β2, HEK293 cells were co-transfected with pLTCCβ-GFP and equimolar ratios of the vector plasmids PPT.H1.β2 or PPT.H1.NS. Transfections were performed using Lipofectamine 2000 reagent as per manufacturers instructions (Invitrogen). Twenty-four hours after transfection, β2-GFP expression was established by fluorescence microscopy and quantified by flow cytometric analysis (Becton Dickinson).

### Primary Culture of Neonatal Rat Cardiac Ventricular Myocytes

Neonatal rat cardiac myocytes (NRCMs) were isolated from 1-2 day old Sprague-Dawley rats and cultured as previously described^{15,16}. Hearts were removed and ventricles minced in calcium- and bicarbonate-free Hanks' buffer with HEPES. These tissue fragments were digested by stepwise trypsin dissociation. In order to diminish the amount of fibroblasts in the culture, the dissociated cells were pre-plated for 45 minutes. Non-adherent myocytes were plated at a density of 1300 cells/mm² in plating medium consisting of DMEM (Mediatech) supplemented with 5% FBS, penicillin (100 U/mL), streptomycin (100 mg/mL), and 2 μg/mL vitamin B₁₂. The cells were maintained at 37° C. in the presence of 5% CO₂ in a humidified incubator. Bromodeoxyuridine (0.1 mmol/L) was added in the medium for the first 72 hours after isolation to inhibit fibroblast growth. For hypertrophy experiments cells were placed in serum-free DMEM containing 3.8 g/L glucose, vitamin B₁₂, transferrin, and insulin 24 hours before phenylephrine (PE) stimulation.

### Single Cell Electrophysiology.

NRCMs were plated at low density (100 cells/mm²) on laminin-coated 12 mm glass cover slips. Rhodamine-tagged siRNAs against the LTCCβ and the NS control were transfected using RNA-Ifect transfection reagent as per manufacturer's instructions (Qiagen). Membrane currents were recorded 72 hours after transfection using the whole-cell patch clamp technique with an Axopatch 200B amplifier (Axon Instruments). Borosilicate glass pipettes were pulled and fire-polished to final tip resistances of 1.5-2.5 MΩ when filled with recording solution. Cells transfected with rhodamine-tagged siRNAs were recognized by fluorescent microscopy (FIG. 1 B). All recordings were performed at room temperature. Cells were superfused in solution containing (in mmol/L): 140 NaCl, 5 KCl, 1 MgCl₂, 10 HEPES, 2 CaCl₂, and 10 glucose (pH 7.4 adjusted with NaOH). After establishing the whole-cell patch clamp mode, the external solution was replaced with solution containing (mmol/L): 130 NaCl, 5 KCl, 1 MgCl₂, 10 HEPES, 5 CsCL₂, 15 BaCL₂, and 10 glucose (pH 7.4 adjusted with CsOH). The pipette electrode solution for I_{CaL} was composed of (in mmol/L): 110 CsCl, 20 TEA, 10 HEPES, 5 BAPTA, 5 Mg-ATP, 1 MgCl₂, and 5 glucose (pH 7.2 adjusted with CsOH). L-type calcium currents were elicited by 300 ms-depolarizing steps from −40 to 60 mV in 10 mV increments. To inactivate Na current, a pre-pulse from −80 mV to −40 mV was used.

### Calcium Transients

NRCMs were plated into 35 mm glass bottom dishes (MatTek Cultureware) and analysed 72 hours after transduction. Cells were loaded with Rhod2-AM (2 μM) (Molecular Probes) for 18 minutes. Following this cells were washed with PBS and placed in phenol-free Modified Eagle medium (GIBCO/Invitrogen). Calcium transients were measured at 37° C. during field stimulation at 1.5 Hz to ensure consistent diastolic intervals. Images were acquired on an inverted confocal laser-scanning microscope (Perkin Elmer/Nikon). Transduced cells, recognized by GFP fluorescence, were randomly selected for recording of calcium transients. These were subsequently analyzed using image-J software (NIH).

### Cell Area and [³H]Leucine Incorporation.

For cell area measurements, NRCMs were plated at low density (100 cells/mm²) in 35 mm glass-bottom dishes. Cells were serum starved for 24 hours, after which they were incubated in phenylephrine (PE), 10 uM. Images were acquired on an inverted microscope (Nikon) and cell area measurements were performed offline using NIH's Image-J software. Transduced cells were recognized by GFP fluorescence.

For [³H]leucine incorporation, cells were plated in 12 well plates at a density of 0.5×10⁶ cells/well. Seventy two hours after transduction cells were incubated for 24 hours with 2 μCi/ml of [3H]-leucine (MP Biomedicals) with and without PE. After incubation, cells were washed with ice-cold PBS, and fixed with 10% trichloroacetic acid for 30 minutes. Cell lysates were then solubilized in 0.20 N NaOH and the incorporated radioactivity was determined by liquid scintillation counting.

### In Vivo Cardiac Gene Transfer and Aortic Banding

Adult (240-260 g) Sprague-Dawley rats, were randomly assigned to receive PPTCG.H1.B2, PPTCG.H1.NS, or sham intervention. After baseline echocardiographic recordings, rats were anesthetized with isoflurane, intubated and placed on a volume-cycled mechanical ventilator. Body temperature was monitored and kept constant at 37° C. throughout the procedure. After dissection of the aorta and pulmonary artery, lentivirus vector (200 ul=10×10⁸ TU/heart) was injected into the LV cavity through a 28 G needle syringe while the aorta and pulmonary artery were cross-clamped for 50 seconds. Fifteen minutes after the aorto/pulmonary cross clamp was released, the ascending aorta was banded with a 0.58 mm (internal diameter) tantalum clip as previously described.¹⁷. In sham operated animals, 150 ul of normal saline was injected into the LV cavity while the aorta and pulmonary artery were cross-clamped for 50 seconds. After cross-clamping was released the chest was closed, and no aortic banding was performed. In vivo transduction efficiency was assessed by X-Gal staining of PPT.CMV-LacZnls injected animals. After 4 weeks of gene delivery, hearts were extracted and whole heart fixation and X-Gal staining was performed by retrograde perfusion as previously described¹⁸. Paraffin-embedded tissue sections (15 μm) were deparaffinated, stained with Hoechst® nuclear staining and mounted. Same field (20×) images were acquired by fluorescence and light microscopy and subsequently analyzed using Image J software.

### Echocardiography.

Rats were anesthetized with ketamine HCL (50 mg/kg IP) and xylazine (20 mg/kg IP), shaved over the praecordium and placed on a rodent-handling platform (VisualSonics) that allowed electrocardiography (ECG) monitoring and body temperature to be kept constant at 37° C. Transthoracic 2-D and M-mode images were acquired on the para-sternal long-axis and short-axis at the midpapillary level using a high-resolution 25 MHZ scan head (RMV-710, VisualSonics) attached to a rail system to assure standardization of imaging acquisition between animals. All images were recorded in a VisualSonics Vevo 660 high resolution rodent imaging system, and subsequently analyzed. Echocardiograms were performed at baseline, 2 and 4-week time points after vector transduction/aortic banding. LV wall thickness and LV end diastolic (LVDD) and end systolic (LVSD) diameters were measured offline from M-mode recordings. Fractional shortening (%) was calculated as 100×(LVDD−LVSD)/LVDD. Statistics.

Continuous variables are expressed as mean±standard error of the mean. Statistical analyses were performed using repeated measures ANOVA and Student paired t test, where appropriate. p<0.05 was considered to be indicative of statistical significance.

### EXAMPLE 2

### Effect of shRNA on LTCCβ Expression

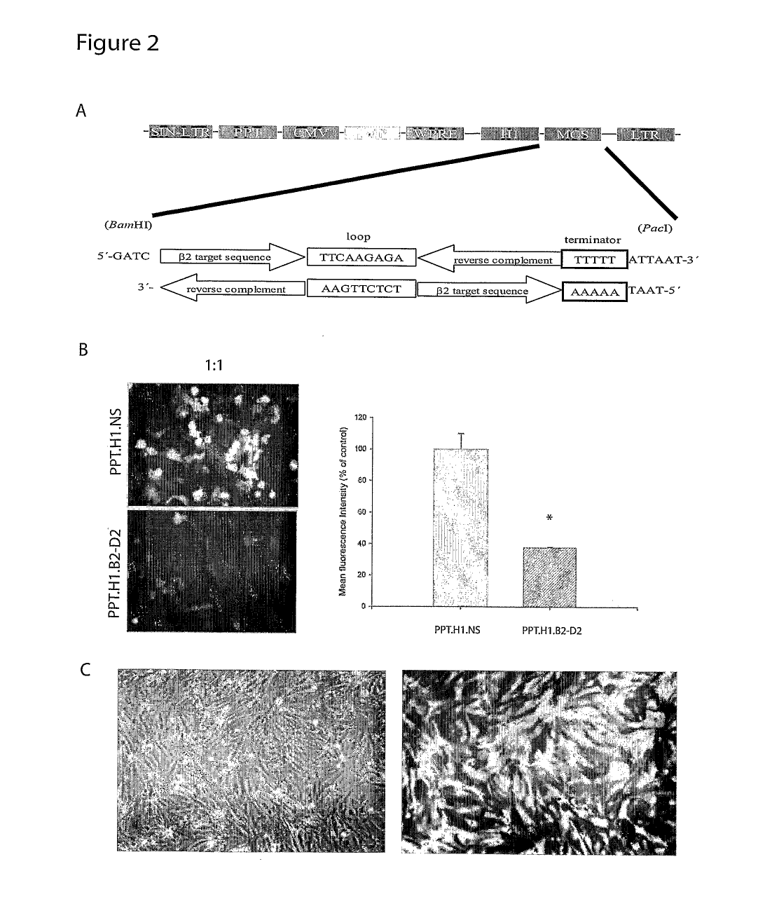

The gene-silencing capacity of our shRNA was initially tested in a heterologous expression system. Twenty four hours after transfection with p.LTCCβ-GFP, ˜85% of HEK293 cells were positive for GFP fluorescence. As evidenced by fluorescent microscopy, co-transfection of PPT.H1.β₂ significantly decreased GFP expression relative to cells co-transfected with PPT.H1.NS. (FIG. 2-B, left panel). In order to quantify the gene silencing efficacy of our construct, cells were subjected to FACS analysis. In two separate experiments, PT.H1.β₂ reduced the mean fluorescence intensity of pLTCCβ-GFP cotransfected cells by 64.6±7.5% compared to PPT.H1.NS co-transfected cells (FIG. 2-B, right panel).

### EXAMPLE 3

### Effects of LTCC β₂ Gene Silencing on Calcium Handling

To verify endogenous LTCCβ gene silencing, we first recorded calcium currents from NRCMs after transfection of β₂ and NS siRNA duplexes. Barium was used as a surrogate charge carrier in order to maximize the resolution of ionic currents. Peak current density was dramatically reduced in β2 siRNA transduced cells (at 10 mV−2.80±0.82 pA/pF, n=4) compared to NS siRNA transduced controls (at 10 mV−33.02±10.9 pA/pF, n=3, p<0.05) (FIG. 1).

Suppression of LTCC would be predicted to attenuate intracellular calcium cycling. For calcium transient recordings, NRCMs were transduced with either PPT.CG.H1.B2 or PPT.CG.H1.NS at a multiplicity of infectivity (MOI) of 50 to achieve a transduction efficiency greater than 90% when determined by fluorescence microscopy (FIG. 2.C).

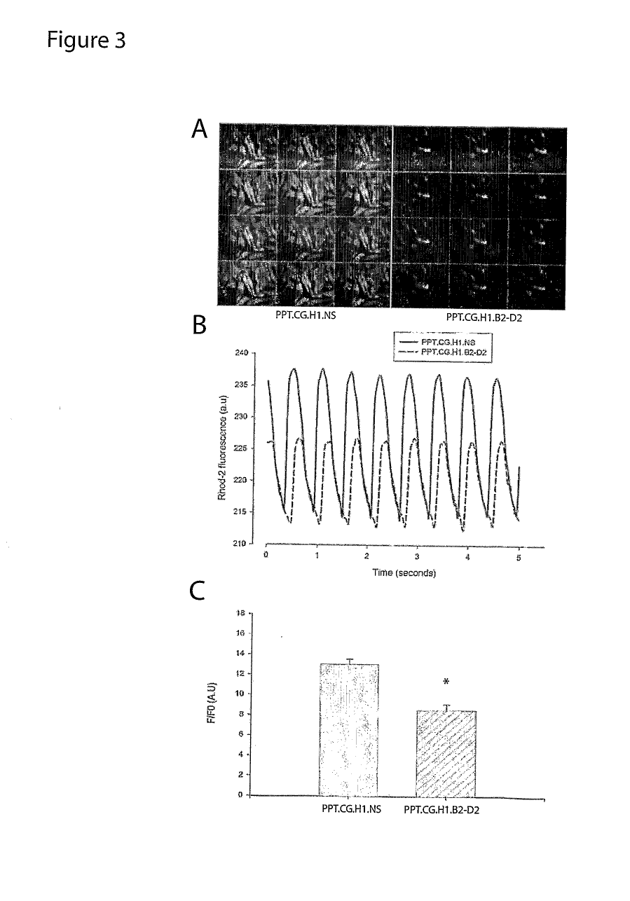

Seventy two hours later, PPT.CG.H1.B2-transduced cells (n=101) showed a reduced calcium transient amplitude of 34% compared to PPT.CG.H1.NS (n=102) transduced controls. (F/Fo=8.54±0.61 vs. F/Fo=13.05±0.55, p<0.01). (FIG. 3).

### EXAMPLE 4

### Effect of LTCCβ₂ Gene Knock Down on Cardiac Hypertrophy In Vitro

The role of post-transcriptional gene silencing of LTCC β₂ was assessed in a PE-induced NRCM hypertrophy model. 72 hours after transduction (MOI of 50) cells were serum starved for 24 hours and PE stimulation was initiated. PPT.CG.H1.B2 transduced cells (n=19) showed a 36% decrease in cell size compared to PPT.CG.H1.NS transduced controls (n=31). (p<0.05) after 48 hours of PE stimulation. (FIG. 3.A,B). Given that cultured cells are subjected to changes in cell volume and area not necessarily related to the development of hypertrophy, [³H]leucine incorporation was measured after 24 hours stimulation with PE. Interestingly, PE-stimulated [³H]leucine incorporation was suppressed in PPT.CG.H1.B2 transduced cells (99.3±13% of control, n=9) compared to PPT.CG.H1.NS transduced cells (173.6±18% of control, n=9). (p<0.05). (FIG. 3.C).

### EXAMPLE 5

### Effect of LTCC β₂ Gene Knock Down on Cardiac Hypertrophy In Vivo

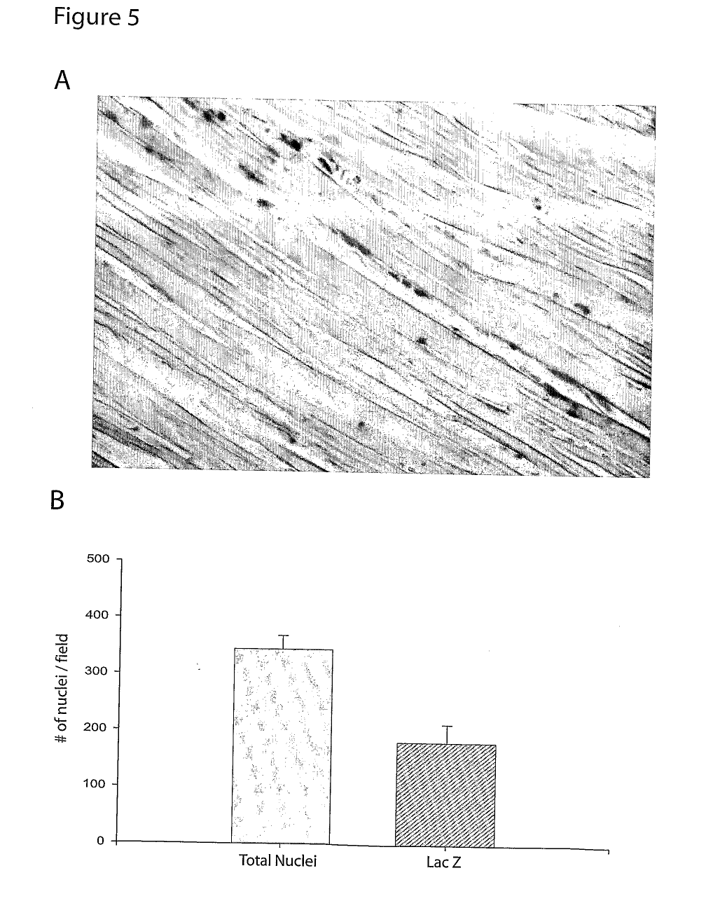

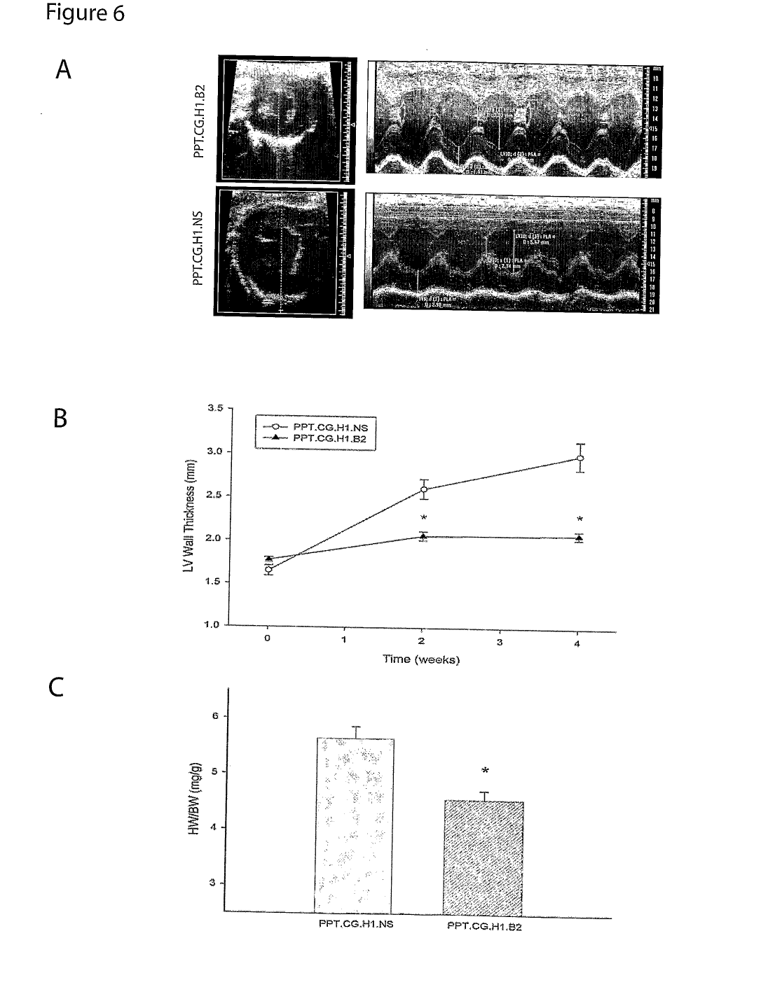

We and others have previously shown the efficacy of LV injection and aorto-pulmonary cross clamping for in vivo cardiac adenoviral vector-mediated gene delivery.^{19,20}. Recently, advanced-generation lentiviral vectors have also been reported to efficiently transduce the rat heart by the same vector delivery technique²¹. We therefore implemented a banding model of LVH in the rat and compared three groups: sham-operated, beta-suppressive, and non-silencing RNA vector groups. The transduction efficiency achieved in the current study was ˜50% as established by X-gal staining four weeks after injection of PPT.CMV.LacZnls (FIG. 5). No significant differences in baseline characteristics were observed between the three study groups (Table. 1). During follow up, PPT.CG.H1.B2 transduced animals exhibited an attenuated hypertrophy response compared to non-silencing transduced controls. At 30 days after aortic banding, left ventricular wall thickness (LVWT) was decreased by 33% in PPT.CG.H1.B2 transduced rats (n=6) compared to non-silencing controls (n=7). (FIG. 6 A,B and table.1). Moreover, heart weight/body weight ratios (HW/BW), were also decreased in PPT.CG.H1.B2 transduced rats compared to non-silencing controls. (4.55±0.16 vs. 5.64±0.22, p<0.05). (FIG. 6.C and Table.1).

TABLE 1
Baseline and follow up rat data.

| | PPTCG.H1.NS | PPT.CG.H1.β2-D2 | Sham opeated |
|---|---|---|---|
| | (n = 7) | (n = 6) | (n = 4) |
| BW-0 (g) | 261.9 ± 12 | 275.9 ± 16 | 266.4 ± 13 |
| LVWT-0 (mm) | 1.65 ± 0.06 | 1.77 ± 0.02 | 1.67 ± 0.05 |
| SF-0 (%) | 55 ± 3 | 57 ± 4 | 56 ± 2 |
| LVWT-2wks (mm) | 2.61 ± 0.11 | 2.06 ± 0.05 | 1.59 ± 0.3 |
| SF-2 wks (%) | 67 ± 2 | 66 ± 4 | 51 ± 2 |
| LVWT-4 wks (mm) | 2.99 ± 0.16 | 2.08 ± 0.05 (*) | 1.74 ± 0.02 |
| SF-4 wks (%) | 64 ± 2 | 67 ± 4 | 51 ± 1 |
| BW-4 wks (g) | 348.5 ± 11 | 352.6 ± 8 | 353.4 ± 13 |
| HW-4 wks(mg) | 1972.1 ± 120 | 1601.5 ± 47 (*) | 1326.5 ± 94 |
| HW/BW-4 wks (mg/g) | 5.64 ± 0.22 | 4.55 ± 0.16 (*) | 3.73 ± 0.15 |

BW-0: baseline body weight.
LVWT-0: baseline LV wall thickness.
SF-0 baseline shortening fraction.
LVWT-2 wks: 2 weeks LV wall thickness.
SF-2 wks: 2 weeks shortening fraction.
LVWT-4 wks: 4 weeks LV wall thickness.
SF-4 wks: 4 weeks shortening fraction.
BW-4 wks: body weight at 4 weeks.
HW-4 wks: heart weight at 4 weeks.
HW/BW-4 weeks: heart weight/body weight ratio at 4 weeks.
(*) p < 0.05 PPT.CG.H1.B2 compared to non-silencing control

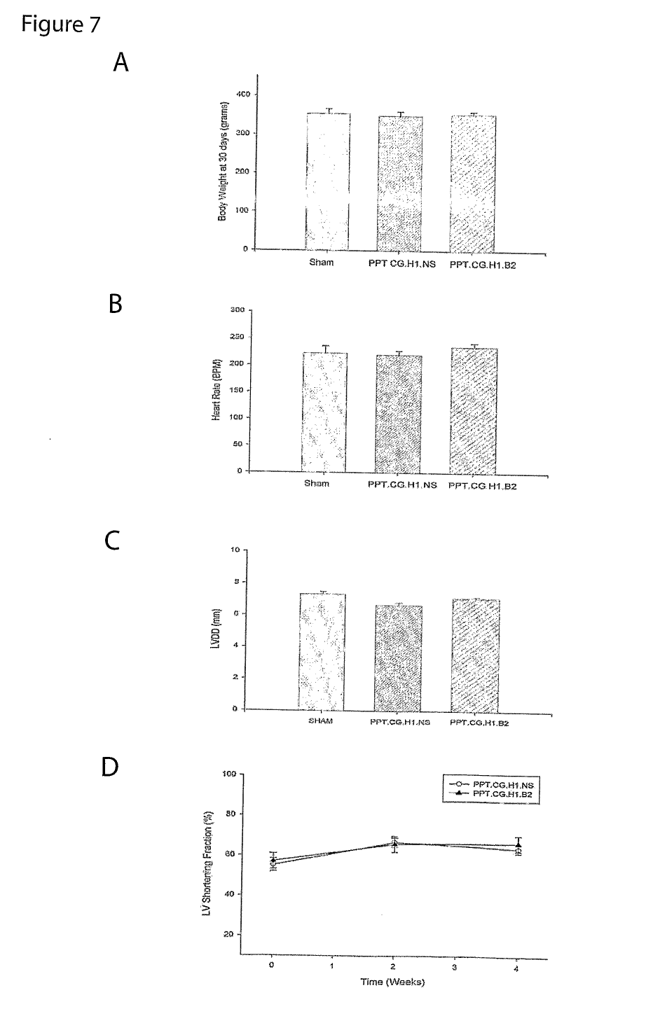

Down regulation of LTCCβ, by affecting calcium influx and release from the intracellular stores, may impair excitation-contraction coupling and systolic function. To exclude this possibility, animals were followed up to detect signs of congestive heart failure and cardiac function was studied non-invasively by high-resolution echocardiography. Interestingly, no signs of respiratory distress, fluid retention, or differences in body weight, ventricular heart rate were detected between groups. (FIG. 7.A,B and table.1). Moreover, during follow-up, left ventricular diastolic diameter and shortening fraction were comparable in PPT.CG.H1.B2 and PPT.CG.H1.NS transduced rats. (FIG. 7.C,D and table.1).

### REFERENCES

The disclosure of each reference cited is expressly incorporated herein.
1. Schulman S P, Weiss J L, Becker L C, Gottlieb S O, Woodruff K M, Weisfeldt M L, Gerstenblith G. The effects of antihypertensive therapy on left ventricular mass in elderly patients. N Engl J Med. 1990; 322:1350-6.
2. Levy D, Garrison R J, Savage D D, Kannel W B, Castelli W P. Prognostic implications of echocardiographically determined left ventricular mass in the Framingham Heart Study. N Engl J Med. 1990; 322:1561-6.
3. Molkentin J D, Lu J R, Antos C L, Markham B, Richardson J, Robbins J, Grant S R, Olson E N. A calcineurin-dependent transcriptional pathway for cardiac hypertrophy. Cell. 1998; 93:215-28.
4. Bers D M. Cardiac excitation-contraction coupling. Nature. 2002; 415:198-205.
5. Klingbeil A U, Schneider M, Martus P, Messerli F H, Schmieder R E. A meta-analysis of the effects of treatment on left ventricular mass in essential hypertension. Am J Med. 2003; 115:41-6.
6. Chien A J, Hosey M M. Post-translational modifications of beta subunits of voltage-dependent calcium channels. J Bioenerg Biomembr. 1998; 30:377-86.
7. Chien A J, Carr K M, Shirokov R E, Rios E, Hosey M M. Identification of palmitoylation sites within the L-type calcium channel beta2a subunit and effects on channel function. J Biol Chem. 1996; 271:26465-8.
8. Van Petegem F, Clark K A, Chatelain F C, Minor D L, Jr. Structure of a complex between a voltage-gated calcium channel beta-subunit and an alpha-subunit domain. Nature. 2004; 429:671-5.
9. Chen Y H, Li M H, Zhang Y, He L L, Yamada Y, Fitzmaurice A, Shen Y, Zhang H, Tong L, Yang J. Structural basis of the alpha1-beta subunit interaction of voltage-gated Ca2+ channels. Nature. 2004; 429:675-80.
10. Colecraft H M, Alseikhan B, Takahashi S X, Chaudhuri D, Mittman S, Yegnasubramanian V, Alvania R S, Johns D C, Marban E, Yue D T. Novel functional properties of Ca(2+) channel beta subunits revealed by their expression in adult rat heart cells. J Physiol. 2002; 541:435-52.
11. Reynolds A, Leake D, Boese Q, Scaringe S, Marshall W S, Khvorova A. Rational siRNA design for RNA interference. Nat Biotechnol. 2004; 22:326-30.
12. Kizana E, Ginn S L, Allen D G, Ross D L, Alexander I E. Fibroblasts can be genetically modified to produce excitable cells capable of electrical coupling. Circulation. 2005; 111:394-8.
13. Zufferey R, Donello J E, Trono D, Hope T J. Woodchuck Hepatitis Virus Posttranscriptional Regulatory Element Enhances Expression of Transgenes Delivered by Retroviral Vectors. J. Virol. 1999; 73:2886-2892.
14. Fleming J, Ginn S L, Weinberger R P, Trahair T N, Smythe J A, Alexander I E. Adeno-associated virus and lentivirus vectors mediate efficient and sustained transduction of cultured mouse and human dorsal root ganglia sensory neurons. Hum Gene Ther. 2001; 12:77-86.
15. Akao M, Ohler A, O'Rourke B, Marban E. Mitochondrial ATP-Sensitive Potassium Channels Inhibit Apoptosis Induced by Oxidative Stress in Cardiac Cells. Circ Res. 2001; 88:1267-1275.
16. Johns D C, Nuss H B, Marban E. Suppression of Neuronal and Cardiac Transient Outward Currents by Viral Gene Transfer of Dominant-Negative Kv4.2 Constructs. J. Biol. Chem. 1997; 272:31598-31603.
17. Del Monte F, Butler K, Boecker W, Gwathmey J K, Hajjar R J. Novel technique of aortic banding followed by gene transfer during hypertrophy and heart failure. Physiol Genomics. 2002; 9:49-56.
18. O'Donnell J M, Lewandowski E D. Efficient, cardiac-specific adenoviral gene transfer in rat heart by isolated retrograde perfusion in vivo. Gene Ther. 2005; 12:958-64.
19. Miake J, Marban E, Nuss H B. Functional role of inward rectifier current in heart probed by Kir2.1 overexpression and dominant-negative suppression. J Clin Invest. 2003; 111:1529-36.
20. del Monte F, Hajjar R J. Efficient viral gene transfer to rodent hearts in vivo. Methods Mol Biol. 2003; 219:179-93.
21. Bonci D, Cittadini A, Latronico M V, Borello U, Aycock J K, Drusco A, Inocenzi A, Follenzi A, Lavitrano M, Monti M G, Ross J, Jr., Naldini L, Peschle C, Cossu G, Condorelli G. ‘Advanced’ generation lentiviruses as efficient vectors for cardiomyocyte gene transduction in vitro and in vivo. Gene Ther. 2003; 10:630-6.
22. Feron O, Salomone S, Godfraind T. Action of the calcium channel blocker lacidipine on cardiac hypertrophy and endothelin-1 gene expression in stroke-prone hypertensive rats. Br J Pharmacol. 1996; 118:659-64.
23. Lubic S P, Giacomini K M, Giacomini J C. The effects of modulation of calcium influx through the voltage-sensitive calcium channel on cardiomyocyte hypertrophy. J Mol Cell Cardiol. 1995; 27:917-25.
24. Murata M, Cingolani E, McDonald A D, Donahue J K, Marban E. Creation of a genetic calcium channel blocker by targeted gem gene transfer in the heart. Circ Res. 2004; 95:398-405.
25. Escobar A L, Ribeiro-Costa R, Villalba-Galea C, Zoghbi M E, Perez C G, Mejia-Alvarez R. Developmental changes of intracellular Ca2+ transients in beating rat hearts. Am J Physiol Heart Circ Physiol. 2004; 286:H971-978.
26. Hurtado C, Ander B P, Maddaford T G, Lukas A, Hryshko L V, Pierce G N. Adenovirally delivered shRNA strongly inhibits Na+—Ca2+ exchanger expression but does not prevent contraction of neonatal cardiomyocytes. J Mol Cell Cardiol. 2005; 38:647-54.
27. Hill J A, Karirni M, Kutschke W, Davisson R L, Zimmerman K, Wang Z, Kerber R E, Weiss R M. Cardiac hypertrophy is not a required compensatory response to short-term pressure overload. Circulation. 2000; 101:2863-9.
28. Grossman W, Jones D, McLaurin L P. Wall stress and patterns of hypertrophy in the human left ventricle. J Clin Invest. 1975; 56:56-64.
29. Esposito G, Rapacciuolo A, Naga Prasad S V, Takaoka H, Thomas S A, Koch W J, Rockman H A. Genetic alterations that inhibit in vivo pressure-overload hypertrophy prevent cardiac dysfunction despite increased wall stress. Circulation. 2002; 105:85-92.
30. Chen X, Zhaig X, Kubo H, Harris D M, Mills G D, Moyer J, Berretta R, Potts S T, Marsh J D, Houser S R. Ca2+ influx-induced sarcoplasmic reticulum Ca2+ overload causes mitochondrial-dependent apoptosis in ventricular myocytes. Circ Res. 2005; 97:1009-17.
31. Spirito P, Seidman C E, McKenna W J, Maron B J. The management of hypertrophic cardiomyopathy. N Engl J Med. 1997; 336:775-85.
32. Nielsen C D, Spencer W H, 3rd. Role of controlled septal infarct in hypertrophic obstructive cardiomyopathy. Cardiol Rev. 2002; 10:108-18.
33. Sigwart U. Non-surgical myocardial reduction for hypertrophic obstructive cardiomyopathy. Lancet. 1995; 346:211-4.
34. Chang S M, Nagueh S F, Spencer W H, 3rd, Lakkis N M. Complete heart block: determinants and clinical impact in patients with hypertrophic obstructive cardiomyopathy undergoing nonsurgical septal reduction therapy. J Am Coll Cardiol. 2003; 42:296-300.

1. A method for treating cardiac hypertrophy in a mammal, comprising:
administering to the mammal a vector encoding a short hairpin RNA, said RNA comprising a segment in its double stranded portion that is complementary to an L-type calcium channel accessory β-subunit (LTCCβ) coding sequence, wherein the short hairpin RNA is expressed in the mammal causing one or more physiological effects selected from the group consisting of:
a. reduced peak calcium current density;
b. reduced calcium current amplitude;
c. suppression of phenylephrine-stimulated protein synthesis;
d. suppression of phenylephrine-stimulated cell size increase;
e. decreased cardiac ventricular wall thickness; and
f. attenuated hypertrophy.

2. The method of claim 1 wherein the mammal has hypertrophic obstructive cardiomyopathy.

3. The method of claim 1 wherein the mammal has left ventricular hypertrophy.

4. The method of claim 1 wherein the mammal has hypertrophic cardiomyopathy.

5. The method of claim 1 wherein the vector is a lentivirus vector.

6. The method of claim 1 wherein a chemotherapeutic drug is also administered to the mammal, said chemotherapeutic drug inhibits L-type calcium channels.

7. The method of claim 1 wherein the physiological effect is reduced peak calcium current density.

8. The method of claim 1 wherein the physiological effect is reduced calcium current amplitude.

9. The method of claim 1 wherein the physiological effect is suppression of phenylephrine-stimulated protein synthesis.

10. The method of claim 1 wherein the physiological effect is suppression of phenylephrine-stimulated cell size increase.

11. The method of claim 1 wherein the physiological effect is decreased cardiac ventricular wall thickness.

12. The method of claim 1 wherein the physiological effect is attenuated hypertrophy.

13. The method of claim 7 wherein the peak calcium current density is reduced by at least 10% as determined by a standard electrophysiological assay.

14. The method of claim 8 wherein the calcium current amplitude is reduced by at least 10% as determined by a standard electrophysiological assay.

15. The method of claim 1 wherein the vector is administered directly to the heart.

16. The method of claim 1 wherein the vector is administered directly to the left ventricle.

17. The method of claim 7 wherein the peak calcium current density is reduced by at least 20% as determined by a standard electrophysiological assay.

18. The method of claim 8 wherein the calcium current amplitude is reduced by at least 20% as determined by a standard electrophysiological assay.

19. The method of claim 7 wherein the peak calcium current density is reduced by at least 30% as determined by a standard electrophysiological assay.

20. The method of claim 8 wherein the calcium current amplitude is reduced by at least 30% as determined by a standard electrophysiological assay.

21. The method of claim 1 wherein the segment is in said coding sequence's conserved D2 region.

22. A method for decreasing calcium channel activity in a mammal, comprising:
administering to the mammal a vector encoding a short hairpin RNA, said RNA comprising a segment in its double stranded portion that is complementary to a coding sequence of an L-type calcium channel accessory β-subunit (LTCCβ), wherein the short hairpin RNA is expressed in the mammal causing one or more physiological effects selected from the group consisting of:
a. reduced peak calcium current density;
b. reduced calcium current amplitude;
c. suppression of phenylephrine-stimulated protein synthesis;
d. suppression of phenylephrine-stimulated cell size increase;
e. decreased cardiac ventricular wall thickness; and
f. attenuated hypertrophy.

23. The method of claim 22 wherein the physiological effect is reduced peak calcium current density.

24. The method of claim 22 wherein the physiological effect is reduced calcium current amplitude.

25. The method of claim 22 wherein the physiological effect is suppression of phenylephrine-stimulated protein synthesis.

26. The method of claim 22 wherein the physiological effect is suppression of phenylephrine-stimulated cell size increase.

27. The method of claim 22 wherein the physiological effect is decreased cardiac ventricular wall thickness.

28. The method of claim 22 wherein the physiological effect is attenuated hypertrophy.

29. The method of claim 22 wherein the vector is a lentivirus vector.

30. The method of claim 21 wherein the segment is in said coding sequence's conserved D2 region.

31. The method of claim 1 or 221 wherein the segment has the sequence: AAC ATG AGG CTA CAG CAT GAA (SEQ ID NO: 10).


SEQUENCE LISTING (10 sequences)

SEQ ID NO: 1 - DNA, 3373 bp, Homo sapiens
gaggaggaggggacccgccgccgggggctggctgcttcgctccgagccgacttttcgccaatggtccaaagggacatgtccaagtcgcctcccacagcggcggcggcggtggcgcaggagatccagatggaactgctagagaacgtggctcccgcgggggcgctcggagccgccgcacagtcatatggaaaaggagccagaaggaaaaacagatttaaaggatctgatggaagcacgtcatctgatactacctcaaatagttttgttcgccagggttcggcagactcctacactagccgtccatccgattccgatgtatctctggaggaggaccgggaggcagtgcgcagagaagcggagcggcaggcccaggcacagttggaaaaagcaaagacaaagcccgttgcatttgcggttcggacaaatgtcagctacagtgcggcccatgaagatgatgttccagtgcctggcatggccatctcattcgaagcaaaagattttctgcatgttaaggaaaaatttaacaatgactggtggatagggcgattggtaaaagaaggctgtgaaatcggattcattccaagcccagtcaaactagaaaacatgaggctgcagcatgaacagagagccaagcaagggaaattctactccagtaaatcaggaggaaattcatcatccagtttgggtgacatagtacctagttccagaaaatcaacacctccatcatctggtgcaaaatctgcagatgaacaagaccagtggaaaactgcaggcttgttttggcggtttactacagagcacactcctccgtatgatgtggtaccttccatgcgaccagtggtcctagtgggcccttctctgaagggctacgaggtcacagatatgatgcaaaaagcgctgtttgattttttaaaacacagatttgaagggcggatatccatcacaagggtcaccgctgacatctcgcttgccaaacgctcggtattaaacaatcccagtaagcacgcaataatagaaagatccaacacaaggtcaagcttagcggaagttcagagtgaaatcgaaaggatttttgaacttgcaagaacattgcagttggtggtccttgacgcggatacaattaatcatccagctcaactcagtaaaacctccttggcccctattatagtatatgtaaagatttcttctcctaaggttttacaaaggttaataaaatctcgagggaaatctcaagctaaacacctcaacgtccagatggtagcagctgataaactggctcagtgtcctccagagctgttcgatgtgatcttggatgagaaccagcttgaggatgcctgtgagcaccttgccgactatctggaggcctactggaaggccacccatcctcccagcagtagcctccccaaccctctccttagccgtacattagccacttcaagtctgcctcttagccccaccctagcctctaattcacagggttctcaaggtgatcagaggactgatcgctccgctcctatccgttctgcttcccaagctgaagaagaacctagtgtggaaccagtcaagaaatcccagcaccgctcttcctcctcagccccacaccacaaccatcgcagtgggacaagtcgcggcctctccaggcaagagacatttgactcggaaacccaggagagtcgagactctgcctacgtagagccaaaggaagattattcccatgaccacgtggaccactatgcctcacaccgtgaccacaaccacagagacgagacccacgggagcagtgaccacagacacagggagtcccggcaccgttcccgggacgtggatcgagagcaggaccacaacgagtgcaacaagcagcgcagccgtcataaatccaaggatcgctactgtgaaaaggatggagaagtgatatcaaaaaaacggaatgaggctggggagtggaacagggatgtttacatccgccaatgagttttgcccttttgtgtttttttttttttttttttgaagtcttgtataactaacagcatccccaaaacaaagtctttggggtctacactgcaatcatatgtgatctgtcttgtaatattttgtattattgctgttgcttgaatagcaatagcatggatagagtattgagatactttttcttttgtaagtgctacataaattggcctggtatggctgcagtcctccggttgcatactggactcttcaaaaactgttttgggtagctgccacttgaacaaaatctgttgccacccaggtgatgttagtgttttaagaaatgtagttgatgtatccaacaagccagaatcagcacagataaaaagtggaatttcttgtttctccagatttttaatacgttaatacgcaggcatctgatttgcatattcattcatggaccactgtttcttgcttgtacctctggctgactaaatttggggacagattcagtcttgccttacacaaaggggatcataaagttagaatctattttctatgtactagtactgtgtactgtatagacagtttgtaaatgttatttctgcaaacaaacaccttcttattatatataatatatatatatatatcagtttgatcacactattttagagtcttaatgccaagtcagcagatttgctttatgaattacagggactagaaatgcccacattcaggaaatttgtaataacattgtctagacacctatcctcattctagtagaaagtgtgtacatactgtaaatatgtgtgattgcttgacttgaaaaggtttgaattctgaatgttataccatccttgtaagtaagtttgtaatttccaccataaattatggtaaatataaaactccagaggttgttctactccatacagttcacactgattgtgacacattcttagtagctagtgtctgttctagtcactgcactggagtctacgagccggaactcgctatatgcacgtgtgtgtgtccgtatgtaagaaagtgtgcaccgagtgactgaatggttgagatgaattggaatgctgaagactaacgaagaaactagagactgatatcgagcattctgcccacctcgctctgtatttaattaattgtgctatatgttgctttaacaacccattgagcagtcagggaatgtgagtaagcttgctgccaaaggtaactaggaaagcattcatctgctgcctccttgtttttgctcctagagagtgaaaatacaggcaattttactgtgagtgtttcactggaaatgtacaatctttgtgtgttagagtatttgttttagtaagaaatgtttacacagcttgtggaattatttcgtgggaaaataaatttttataacttctcccaaaaaaaaaaaaaaaaa

SEQ ID NO: 2 - DNA, 3375 bp, Homo sapiens
gactacacaccccaagccagcaagaaaaaggaaagcagttgctatgctgttcagcaaagcaagacttggctgccttttagctagtcctgaattcttgctcctgtgaagaaaattcctgctggagtgctgggcgcacttggaattggtctagcatgcttgacagacgccttatagctcctcaaactaaatacattattcctgggggttcggcagactcctacactagccgtccatccgattccgatgtatctctggaggaggaccgggaggcagtgcgcagagaagcggagcggcaggcccaggcacagttggaaaaagcaaagacaaagcccgttgcatttgcggttcggacaaatgtcagctacagtgcggcccatgaagatgatgttccagtgcctggcatggccatctcattcgaagcaaaagattttctgcatgttaaggaaaaatttaacaatgactggtggatagggcgattggtaaaagaaggctgtgaaatcggattcattccaagcccagtcaaactagaaaacatgaggctgcagcatgaacagagagccaagcaagggaaattctactccagtaaatcaggaggaaattcatcatccagtttgggtgacatagtacctagttccagaaaatcaacacctccatcatctgctatagacatagatgctactggcttagatgcagaagaaaatgatattccagcaaaccaccgctcccctaaacccagtgcaaacagtgtaacgtcaccccactccaaagagaaaagaatgcccttctttaagaagacagagcacactcctccgtatgatgtggtaccttccatgcgaccagtggtcctagtgggcccttctctgaagggctacgaggtcacagatatgatgcaaaaagcgctgtttgattttttaaaacacagatttgaagggcggatatccatcacaagggtcaccgctgacatctcgcttgccaaacgctcggtattaaacaatcccagtaagcacgcaataatagaaagatccaacacaaggtcaagcttagcggaagttcagagtgaaatcgaaaggatttttgaacttgcaagaacattgcagttggtggtccttgacgcggatacaattaatcatccagctcaactcagtaaaacctccttggcccctattatagtatatgtaaagatttcttctcctaaggttttacaaaggttaataaaatctcgagggaaatctcaagctaaacacctcaacgtccagatggtagcagctgataaactggctcagtgtcctccagagctgttcgatgtgatcttggatgagaaccagcttgaggatgcctgtgagcaccttgccgactatctggaggcctactggaaggccacccatcctcccagcagtagcctccccaaccctctccttagccgtacattagccacttcaagtctgcctcttagccccaccctagcctctaattcacagggttctcaaggtgatcagaggactgatcgctccgctcctatccgttctgcttcccaagctgaagaagaacctagtgtggaaccagtcaagaaatcccagcaccgctcttcctcctcagccccacaccacaaccatcgcagtgggacaagtcgcggcctctccaggcaagagacatttgactcggaaacccaggagagtcgagactctgcctacgtagagccaaaggaagattattcccatgaccacgtggaccactatgcctcacaccgtgaccacaaccacagagacgagacccacgggagcagtgaccacagacacagggagtcccggcaccgttcccgggacgtggatcgagagcaggaccacaacgagtgcaacaagcagcgcagccgtcataaatccaaggatcgctactgtgaaaaggatggagaagtgatatcaaaaaaacggaatgaggctggggagtggaacagggatgtttacatcccccaatgagttttgcccttttgtgtttttttttttttttttttgaagtcttgtataactaacagcatccccaaaacaaagtctttggggtctacactgcaatcatatgtgatctgtcttgtaatattttgtattattgctgttgcttgaatagcaatagcatggatagagtattgagatactttttcttttgtaagtgctacataaattggcctggtatggctgcagtcctccggttgcatactggactcttcaaaaactgttttgggtagctgccacttgaacaaaatctgttgccacccaggtgatgttagtgttttaagaaatgtagttgatgtatccaacaagccagaatcagcacagataaaaagtggaatttcttgtttctccagatttttaatacgttaatacgcaggcatctgatttgcatattcattcatggaccactgtttcttgcttgtacctctggctgactaaatttggggacagattcagtcttgccttacacaaaggggatcataaagttagaatctattttctatgtactagtactgtgtactgtatagacagtttgtaaatgttatttctgcaaacaaacacctccttattatatataatatatatatatatatcagtttgatcacactattttagagtcttaatgccaagtcagcagatttgctttatgaattacagggactagaaatgcccacattcaggaaatttgtaataacattgtctagacacctatcctcattctagtagaaagtgtgtacatactgtaaatatgtgtgattgcttgacttgaaaaggtttgaattctgaatgttataccatccttgtaagtaagtttgtaatttccaccataaattatggtaaatataaaactccagaggttgttctactccatacagttcacactgattgtgacacattcttagtagctagtgtctgttctagtcactgcactggagtctacgagccggaactcgctatatgcacgtgtgtgtgtccgtatgtaagaaagtgtgcaccgagtgactgaatggttgagatgaattggaatgctgaagactaacgaagaaactagagactgatatcgagcattctgcccacctcgctctgtatttaattaattgtgctatatgttgctttaacaacccattgagcagtcagggaatgtgagtaagcttgctgccaaaggtaactaggaaagcattcatctgctgcctccttgtttttgctcctagagagtgaaaatacaggcaattttactgtgagtgtttcactggaaatgtacaatctttgtgtgttagagtatttgttttagtaagaaatgtttacacagcttgtggaattatttcgtgggaaaataaatttttataacttctcccaaaaaaaaaaaaaaaaa

SEQ ID NO: 3 - DNA, 3229 bp, Homo sapiens
atgaatcaggggagtggactggacctgctgaagatctcatatggaaaaggagccagaaggaaaaacagatttaaaggatctgatggaagcacgtcatctgatactacctcaaatagttttgttcgccagggttcggcagactcctacactagccgtccatccgattccgatgtatctctggaggaggaccgggaggcagtgcgcagagaagcggagcggcaggcccaggcacagttggaaaaagcaaagacaaagcccgttgcatttgcggttcggacaaatgtcagctacagtgcggcccatgaagatgatgttccagtgcctggcatggccatctcattcgaagcaaaagattttctgcatgttaaggaaaaatttaacaatgactggtggatagggcgattggtaaaagaaggctgtgaaatcggattcattccaagcccagtcaaactagaaaacatgaggctgcagcatgaacagagagccaagcaagggaaattctactccagtaaatcaggaggaaattcatcatccagtttgggtgacatagtacctagttccagaaaatcaacacctccatcatctggtgcaaaatctgcagatgaacaagaccagtggaaaactgcaggcttgttttggcggtttactacagagcacactcctccgtatgatgtggtaccttccatgcgaccagtggtcctagtgggcccttctctgaagggctacgaggtcacagatatgatgcaaaaagcgctgtttgattttttaaaacacagatttgaagggcggatatccatcacaagggtcaccgctgacatctcgcttgccaaacgctcggtattaaacaatcccagtaagcacgcaataatagaaagatccaacacaaggtcaagcttagcggaagttcagagtgaaatcgaaaggatttttgaacttgcaagaacattgcagttggtggtccttgacgcggatacaattaatcatccagctcaactcagtaaaacctccttggcccctattatagtatatgtaaagatttcttctcctaaggttttacaaaggttaataaaatctcgagggaaatctcaagctaaacacctcaacgtccagatggtagcagctgataaactggctcagtgtcctccagagctgttcgatgtgatcttggatgagaaccagcttgaggatgcctgtgagcaccttgccgactatctggaggcctactggaaggccacccatcctcccagcagtagcctccccaaccctctccttagccgtacattagccacttcaagtctgcctcttagccccaccctagcctctaattcacagggttctcaaggtgatcagaggactgatcgctccgctcctatccgttctgcttcccaagctgaagaagaacctagtgtggaaccagtcaagaaatcccagcaccgctcttcctcctcagccccacaccacaaccatcgcagtgggacaagtcgcggcctctccaggcaagagacatttgactcggaaacccaggagagtcgagactctgcctacgtagagccaaaggaagattattcccatgaccacgtggaccactatgcctcacaccgtgaccacaaccacagagacgagacccacgggagcagtgaccacagacacagggagtcccggcaccgttcccgggacgtggatcgagagcaggaccacaacgagtgcaacaagcagcgcagccgtcataaatccaaggatcgctactgtgaaaaggatggagaagtgatatcaaaaaaacggaatgaggctggggagtggaacagggatgtttacatcccccaatgagttttgcccttttgtgtttttttttttttttttttgaagtcttgtataactaacagcatccccaaaacaaagtctttggggtctacactgcaatcatatgtgatctgtcttgtaatattttgtattattgctgttgcttgaatagcaatagcatggatagagtattgagatactttttcttttgtaagtgctacataaattggcctggtatggctgcagtcctccggttgcatactggactcttcaaaaactgttttgggtagctgccacttgaacaaaatctgttgccacccaggtgatgttagtgttttaagaaatgtagttgatgtatccaacaagccagaatcagcacagataaaaagtggaatttcttgtttctccagatttttaatacgttaatacgcaggcatctgatttgcatattcattcatggaccactgtttcttgcttgtacctctggctgactaaatttggggacagattcagtcttgccttacacaaaggggatcataaagttagaatctattttctatgtactagtactgtgtactgtatagacagtttgtaaatgttatttctgcaaacaaacacctccttattatatataatatatatatatatatcagtttgatcacactattttagagtcttaatgccaagtcagcagatttgctttatgaattacagggactagaaatgcccacattcaggaaatttgtaataacattgtctagacacctatcctcattctagtagaaagtgtgtacatactgtaaatatgtgtgattgcttgacttgaaaaggtttgaattctgaatgttataccatccttgtaagtaagtttgtaatttccaccataaattatggtaaatataaaactccagaggttgttctactccatacagttcacactgattgtgacacattcttagtagctagtgtctgttctagtcactgcactggagtctacgagccggaactcgctatatgcacgtgtgtgtgtccgtatgtaagaaagtgtgcaccgagtgactgaatggttgagatgaattggaatgctgaagactaacgaagaaactagagactgatatcgagcattctgcccacctcgctctgtatttaattaattgtgctatatgttgctttaacaacccattgagcagtcagggaatgtgagtaagcttgctgccaaaggtaactaggaaagcattcatctgctgcctccttgtttttgctcctagagagtgaaaatacaggcaattttactgtgagtgtttcactggaaatgtacaatctttgtgtgttagagtatttgttttagtaagaaatgtttacacagcttgtggaattatttcgtgggaaaataaatttttataacttctcccaaaaaaaaaaaaaaaaa

SEQ ID NO: 4 - DNA, 3301 bp, Homo sapiens
atgaatcaggggagtggactggacctgctgaagatctcatatggaaaaggagccagaaggaaaaacagatttaaaggatctgatggaagcacgtcatctgatactacctcaaatagttttgttcgccagggttcggcagactcctacactagccgtccatccgattccgatgtatctctggaggaggaccgggaggcagtgcgcagagaagcggagcggcaggcccaggcacagttggaaaaagcaaagacaaagcccgttgcatttgcggttcggacaaatgtcagctacagtgcggcccatgaagatgatgttccagtgcctggcatggccatctcattcgaagcaaaagattttctgcatgttaaggaaaaatttaacaatgactggtggatagggcgattggtaaaagaaggctgtgaaatcggattcattccaagcccagtcaaactagaaaacatgaggctgcagcatgaacagagagccaagcaagggaaattctactccagtaaatcaggaggaaattcatcatccagtttgggtgacatagtacctagttccagaaaatcaacacctccatcatctgctatagacatagatgctactggcttagatgcagaagaaaatgatattccagcaaaccaccgctcccctaaacccagtgcaaacagtgtaacgtcaccccactccaaagagaaaagaatgcccttctttaagaagacagagcacactcctccgtatgatgtggtaccttccatgcgaccagtggtcctagtgggcccttctctgaagggctacgaggtcacagatatgatgcaaaaagcgctgtttgattttttaaaacacagatttgaagggcggatatccatcacaagggtcaccgctgacatctcgcttgccaaacgctcggtattaaacaatcccagtaagcacgcaataatagaaagatccaacacaaggtcaagcttagcggaagttcagagtgaaatcgaaaggatttttgaacttgcaagaacattgcagttggtggtccttgacgcggatacaattaatcatccagctcaactcagtaaaacctccttggcccctattatagtatatgtaaagatttcttctcctaaggttttacaaaggttaataaaatctcgagggaaatctcaagctaaacacctcaacgtccagatggtagcagctgataaactggctcagtgtcctccagagctgttcgatgtgatcttggatgagaaccagcttgaggatgcctgtgagcaccttgccgactatctggaggcctactggaaggccacccatcctcccagcagtagcctccccaaccctctccttagccgtacattagccacttcaagtctgcctcttagccccaccctagcctctaattcacagggttctcaaggtgatcagaggactgatcgctccgctcctatccgttctgcttcccaagctgaagaagaacctagtgtggaaccagtcaagaaatcccagcaccgctcttcctcctcagccccacaccacaaccatcgcagtgggacaagtcgcggcctctccaggcaagagacatttgactcggaaacccaggagagtcgagactctgcctacgtagagccaaaggaagattattcccatgaccacgtggaccactatgcctcacaccgtgaccacaaccacagagacgagacccacgggagcagtgaccacagacacagggagtcccggcaccgttcccgggacgtggatcgagagcaggaccacaacgagtgcaacaagcagcgcagccgtcataaatccaaggatcgctactgtgaaaaggatggagaagtgatatcaaaaaaacggaatgaggctggggagtggaacagggatgtttacatcccccaatgagttttgcccttttgtgtttttttttttttttttttgaagtcttgtataactaacagcatccccaaaacaaagtctttggggtctacactgcaatcatatgtgatctgtcttgtaatattttgtattattgctgttgcttgaatagcaatagcatggatagagtattgagatactttttcttttgtaagtgctacataaattggcctggtatggctgcagtcctccggttgcatactggactcttcaaaaactgttttgggtagctgccacttgaacaaaatctgttgccacccaggtgatgttagtgttttaagaaatgtagttgatgtatccaacaagccagaatcagcacagataaaaagtggaatttcttgtttctccagatttttaatacgttaatacgcaggcatctgatttgcatattcattcatggaccactgtttcttgcttgtacctctggctgactaaatttggggacagattcagtcttgccttacacaaaggggatcataaagttagaatctattttctatgtactagtactgtgtactgtatagacagtttgtaaatgttatttctgcaaacaaacacctccttattatatataatatatatatatatatcagtttgatcacactattttagagtcttaatgccaagtcagcagatttgctttatgaattacagggactagaaatgcccacattcaggaaatttgtaataacattgtctagacacctatcctcattctagtagaaagtgtgtacatactgtaaatatgtgtgattgcttgacttgaaaaggtttgaattctgaatgttataccatccttgtaagtaagtttgtaatttccaccataaattatggtaaatataaaactccagaggttgttctactccatacagttcacactgattgtgacacattcttagtagctagtgtctgttctagtcactgcactggagtctacgagccggaactcgctatatgcacgtgtgtgtgtccgtatgtaagaaagtgtgcaccgagtgactgaatggttgagatgaattggaatgctgaagactaacgaagaaactagagactgatatcgagcattctgcccacctcgctctgtatttaattaattgtgctatatgttgctttaacaacccattgagcagtcagggaatgtgagtaagcttgctgccaaaggtaactaggaaagcattcatctgctgcctccttgtttttgctcctagagagtgaaaatacaggcaattttactgtgagtgtttcactggaaatgtacaatctttgtgtgttagagtatttgttttagtaagaaatgtttacacagcttgtggaattatttcgtgggaaaataaatttttataacttctcccaaaaaaaaaaaaaaaaa

SEQ ID NO: 5 - DNA, 3241 bp, Homo sapiens
atgaaggccacctggatcaggcttctgaaaagagccaagggaggaaggctgaagaattctgatatctgtggttcggcagactcctacactagccgtccatccgattccgatgtatctctggaggaggaccgggaggcagtgcgcagagaagcggagcggcaggcccaggcacagttggaaaaagcaaagacaaagcccgttgcatttgcggttcggacaaatgtcagctacagtgcggcccatgaagatgatgttccagtgcctggcatggccatctcattcgaagcaaaagattttctgcatgttaaggaaaaatttaacaatgactggtggatagggcgattggtaaaagaaggctgtgaaatcggattcattccaagcccagtcaaactagaaaacatgaggctgcagcatgaacagagagccaagcaagggaaattctactccagtaaatcaggaggaaattcatcatccagtttgggtgacatagtacctagttccagaaaatcaacacctccatcatctgctatagacatagatgctactggcttagatgcagaagaaaatgatattccagcaaaccaccgctcccctaaacccagtgcaaacagtgtaacgtcaccccactccaaagagaaaagaatgcccttctttaagaagacagagcacactcctccgtatgatgtggtaccttccatgcgaccagtggtcctagtgggcccttctctgaagggctacgaggtcacagatatgatgcaaaaagcgctgtttgattttttaaaacacagatttgaagggcggatatccatcacaagggtcaccgctgacatctcgcttgccaaacgctcggtattaaacaatcccagtaagcacgcaataatagaaagatccaacacaaggtcaagcttagcggaagttcagagtgaaatcgaaaggatttttgaacttgcaagaacattgcagttggtggtccttgacgcggatacaattaatcatccagctcaactcagtaaaacctccttggcccctattatagtatatgtaaagatttcttctcctaaggttttacaaaggttaataaaatctcgagggaaatctcaagctaaacacctcaacgtccagatggtagcagctgataaactggctcagtgtcctccagagctgttcgatgtgatcttggatgagaaccagcttgaggatgcctgtgagcaccttgccgactatctggaggcctactggaaggccacccatcctcccagcagtagcctccccaaccctctccttagccgtacattagccacttcaagtctgcctcttagccccaccctagcctctaattcacagggttctcaaggtgatcagaggactgatcgctccgctcctatccgttctgcttcccaagctgaagaagaacctagtgtggaaccagtcaagaaatcccagcaccgctcttcctcctcagccccacaccacaaccatcgcagtgggacaagtcgcggcctctccaggcaagagacatttgactcggaaacccaggagagtcgagactctgcctacgtagagccaaaggaagattattcccatgaccacgtggaccactatgcctcacaccgtgaccacaaccacagagacgagacccacgggagcagtgaccacagacacagggagtcccggcaccgttcccgggacgtggatcgagagcaggaccacaacgagtgcaacaagcagcgcagccgtcataaatccaaggatcgctactgtgaaaaggatggagaagtgatatcaaaaaaacggaatgaggctggggagtggaacagggatgtttacatccgccaatgagttttgcccttttgtgtttttttttttttttttttgaagtcttgtataactaacagcatccccaaaacaaagtctttggggtctacactgcaatcatatgtgatctgtcttgtaatattttgtattattgctgttgcttgaatagcaatagcatggatagagtattgagatactttttcttttgtaagtgctacataaattggcctggtatggctgcagtcctccggttgcatactggactcttcaaaaactgttttgggtagctgccacttgaacaaaatctgttgccacccaggtgatgttagtgttttaagaaatgtagttgatgtatccaacaagccagaatcagcacagataaaaagtggaatttcttgtttctccagatttttaatacgttaatacgcaggcatctgatttgcatattcattcatggaccactgtttcttgcttgtacctctggctgactaaatttggggacagattcagtcttgccttacacaaaggggatcataaagttagaatctattttctatgtactagtactgtgtactgtatagacagtttgtaaatgttatttctgcaaacaaacaccttcttattatatataatatatatatatatatcagtttgatcacactattttagagtcttaatgccaagtcagcagatttgctttatgaattacagggactagaaatgcccacattcaggaaatttgtaataacattgtctagacacctatcctcattctagtagaaagtgtgtacatactgtaaatatgtgtgattgcttgacttgaaaaggtttgaattctgaatgttataccatccttgtaagtaagtttgtaatttccaccataaattatggtaaatataaaactccagaggttgttctactccatacagttcacactgattgtgacacattcttagtagctagtgtctgttctagtcactgcactggagtctacgagccggaactcgctatatgcacgtgtgtgtgtccgtatgtaagaaagtgtgcaccgagtgactgaatggttgagatgaattggaatgctgaagactaacgaagaaactagagactgatatcgagcattctgcccacctcgctctgtatttaattaattgtgctatatgttgctttaacaacccattgagcagtcagggaatgtgagtaagcttgctgccaaaggtaactaggaaagcattcatctgctgcctccttgtttttgctcctagagagtgaaaatacaggcaattttactgtgagtgtttcactggaaatgtacaatctttgtgtgttagagtatttgttttagtaagaaatgtttacacagcttgtggaattatttcgtgggaaaataaatttttataacttctcccaaaaaaaaaaaaaaaaa

SEQ ID NO: 6 - DNA, 3445 bp, Homo sapiens
gaggaggaggggacccgccgccgggggctggctgcttcgctccgagccgacttttcgccaatggtccaaagggacatgtccaagtcgcctcccacagcggcggcggcggtggcgcaggagatccagatggaactgctagagaacgtggctcccgcgggggcgctcggagccgccgcacagtcatatggaaaaggagccagaaggaaaaacagatttaaaggatctgatggaagcacgtcatctgatactacctcaaatagttttgttcgccagggttcggcagactcctacactagccgtccatccgattccgatgtatctctggaggaggaccgggaggcagtgcgcagagaagcggagcggcaggcccaggcacagttggaaaaagcaaagacaaagcccgttgcatttgcggttcggacaaatgtcagctacagtgcggcccatgaagatgatgttccagtgcctggcatggccatctcattcgaagcaaaagattttctgcatgttaaggaaaaatttaacaatgactggtggatagggcgattggtaaaagaaggctgtgaaatcggattcattccaagcccagtcaaactagaaaacatgaggctgcagcatgaacagagagccaagcaagggaaattctactccagtaaatcaggaggaaattcatcatccagtttgggtgacatagtacctagttccagaaaatcaacacctccatcatctgctatagacatagatgctactggcttagatgcagaagaaaatgatattccagcaaaccaccgctcccctaaacccagtgcaaacagtgtaacgtcaccccactccaaagagaaaagaatgcccttctttaagaagacagagcacactcctccgtatgatgtggtaccttccatgcgaccagtggtcctagtgggcccttctctgaagggctacgaggtcacagatatgatgcaaaaagcgctgtttgattttttaaaacacagatttgaagggcggatatccatcacaagggtcaccgctgacatctcgcttgccaaacgctcggtattaaacaatcccagtaagcacgcaataatagaaagatccaacacaaggtcaagcttagcggaagttcagagtgaaatcgaaaggatttttgaacttgcaagaacattgcagttggtggtccttgacgcggatacaattaatcatccagctcaactcagtaaaacctccttggcccctattatagtatatgtaaagatttcttctcctaaggttttacaaaggttaataaaatctcgagggaaatctcaagctaaacacctcaacgtccagatggtagcagctgataaactggctcagtgtcctccagagctgttcgatgtgatcttggatgagaaccagcttgaggatgcctgtgagcaccttgccgactatctggaggcctactggaaggccacccatcctcccagcagtagcctccccaaccctctccttagccgtacattagccacttcaagtctgcctcttagccccaccctagcctctaattcacagggttctcaaggtgatcagaggactgatcgctccgctcctatccgttctgcttcccaagctgaagaagaacctagtgtggaaccagtcaagaaatcccagcaccgctcttcctcctcagccccacaccacaaccatcgcagtgggacaagtcgcggcctctccaggcaagagacatttgactcggaaacccaggagagtcgagactctgcctacgtagagccaaaggaagattattcccatgaccacgtggaccactatgcctcacaccgtgaccacaaccacagagacgagacccacgggagcagtgaccacagacacagggagtcccggcaccgttcccgggacgtggatcgagagcaggaccacaacgagtgcaacaagcagcgcagccgtcataaatccaaggatcgctactgtgaaaaggatggagaagtgatatcaaaaaaacggaatgaggctggggagtggaacagggatgtttacatcccccaatgagttttgcccttttgtgtttttttttttttttttttgaagtcttgtataactaacagcatccccaaaacaaagtctttggggtctacactgcaatcatatgtgatctgtcttgtaatattttgtattattgctgttgcttgaatagcaatagcatggatagagtattgagatactttttcttttgtaagtgctacataaattggcctggtatggctgcagtcctccggttgcatactggactcttcaaaaactgttttgggtagctgccacttgaacaaaatctgttgccacccaggtgatgttagtgttttaagaaatgtagttgatgtatccaacaagccagaatcagcacagataaaaagtggaatttcttgtttctccagatttttaatacgttaatacgcaggcatctgatttgcatattcattcatggaccactgtttcttgcttgtacctctggctgactaaatttggggacagattcagtcttgccttacacaaaggggatcataaagttagaatctattttctatgtactagtactgtgtactgtatagacagtttgtaaatgttatttctgcaaacaaacacctccttattatatataatatatatatatatatcagtttgatcacactattttagagtcttaatgccaagtcagcagatttgctttatgaattacagggactagaaatgcccacattcaggaaatttgtaataacattgtctagacacctatcctcattctagtagaaagtgtgtacatactgtaaatatgtgtgattgcttgacttgaaaaggtttgaattctgaatgttataccatccttgtaagtaagtttgtaatttccaccataaattatggtaaatataaaactccagaggttgttctactccatacagttcacactgattgtgacacattcttagtagctagtgtctgttctagtcactgcactggagtctacgagccggaactcgctatatgcacgtgtgtgtgtccgtatgtaagaaagtgtgcaccgagtgactgaatggttgagatgaattggaatgctgaagactaacgaagaaactagagactgatatcgagcattctgcccacctcgctctgtatttaattaattgtgctatatgttgctttaacaacccattgagcagtcagggaatgtgagtaagcttgctgccaaaggtaactaggaaagcattcatctgctgcctccttgtttttgctcctagagagtgaaaatacaggcaattttactgtgagtgtttcactggaaatgtacaatctttgtgtgttagagtatttgttttagtaagaaatgtttacacagcttgtggaattatttcgtgggaaaataaatttttataacttctcccaaaaaaaaaaaaaaaaa

SEQ ID NO: 7 - DNA, 3721 bp, Homo sapiens
cagcagcgtgctaagaagcagtcacataaacagcagcaggagtaggcctcctgcttttcaaaagcagagtactgcagggtcgcgaaatgcaagacactcagatgtttgaaaatctcccgagttgagaatggctactgtaaaagcgtcaccaagaaactctgacgatctggacagtcctaactctgtgttagcaatacttacttccggaaaattaatgctacttcttgtagatttttgcaaataggaaacccccttgaagaagatctcaaattacgccccccacccccaaaaaaagacaaacaggggagaacaaagttttggcatgcctgcaggaacggtggcttttttagaaactacctaggaggcagaagctaagtgatttgctcatgcctcttacctgggagtagaaggtgggaagaaatggaccgaggctgtgacgagaagacaaggcacagtgcagcttggtgaagccacacgctgactgcgttctgccccctcttcatgcagtgctgcgggctggtgcatcgccggcgagtacgggtgtcctatggttcggcagactcctacactagccgtccatccgattccgatgtatctctggaggaggaccgggaggcagtgcgcagagaagcggagcggcaggcccaggcacagttggaaaaagcaaagacaaagcccgttgcatttgcggttcggacaaatgtcagctacagtgcggcccatgaagatgatgttccagtgcctggcatggccatctcattcgaagcaaaagattttctgcatgttaaggaaaaatttaacaatgactggtggatagggcgattggtaaaagaaggctgtgaaatcggattcattccaagcccagtcaaactagaaaacatgaggctgcagcatgaacagagagccaagcaagggaaattctactccagtaaatcaggaggaaattcatcatccagtttgggtgacatagtacctagttccagaaaatcaacacctccatcatctgctatagacatagatgctactggcttagatgcagaagaaaatgatattccagcaaaccaccgctcccctaaacccagtgcaaacagtgtaacgtcaccccactccaaagagaaaagaatgcccttctttaagaagacagagcacactcctccgtatgatgtggtaccttccatgcgaccagtggtcctagtgggcccttctctgaagggctacgaggtcacagatatgatgcaaaaagcgctgtttgattttttaaaacacagatttgaagggcggatatccatcacaagggtcaccgctgacatctcgcttgccaaacgctcggtattaaacaatcccagtaagcacgcaataatagaaagatccaacacaaggtcaagcttagcggaagttcagagtgaaatcgaaaggatttttgaacttgcaagaacattgcagttggtggtccttgacgcggatacaattaatcatccagctcaactcagtaaaacctccttggcccctattatagtatatgtaaagatttcttctcctaaggttttacaaaggttaataaaatctcgagggaaatctcaagctaaacacctcaacgtccagatggtagcagctgataaactggctcagtgtcctccagagctgttcgatgtgatcttggatgagaaccagcttgaggatgcctgtgagcaccttgccgactatctggaggcctactggaaggccacccatcctcccagcagtagcctccccaaccctctccttagccgtacattagccacttcaagtctgcctcttagccccaccctagcctctaattcacagggttctcaaggtgatcagaggactgatcgctccgctcctatccgttctgcttcccaagctgaagaagaacctagtgtggaaccagtcaagaaatcccagcaccgctcttcctcctcagccccacaccacaaccatcgcagtgggacaagtcgcggcctctccaggcaagagacatttgactcggaaacccaggagagtcgagactctgcctacgtagagccaaaggaagattattcccatgaccacgtggaccactatgcctcacaccgtgaccacaaccacagagacgagacccacgggagcagtgaccacagacacagggagtcccggcaccgttcccgggacgtggatcgagagcaggaccacaacgagtgcaacaagcagcgcagccgtcataaatccaaggatcgctactgtgaaaaggatggagaagtgatatcaaaaaaacggaatgaggctggggagtggaacagggatgtttacatcccccaatgagttttgcccttttgtgtttttttttttttttttttgaagtcttgtataactaacagcatccccaaaacaaagtctttggggtctacactgcaatcatatgtgatctgtcttgtaatattttgtattattgctgttgcttgaatagcaatagcatggatagagtattgagatactttttcttttgtaagtgctacataaattggcctggtatggctgcagtcctccggttgcatactggactcttcaaaaactgttttgggtagctgccacttgaacaaaatctgttgccacccaggtgatgttagtgttttaagaaatgtagttgatgtatccaacaagccagaatcagcacagataaaaagtggaatttcttgtttctccagatttttaatacgttaatacgcaggcatctgatttgcatattcattcatggaccactgtttcttgcttgtacctctggctgactaaatttggggacagattcagtcttgccttacacaaaggggatcataaagttagaatctattttctatgtactagtactgtgtactgtatagacagtttgtaaatgttatttctgcaaacaaacacctccttattatatataatatatatatatatatcagtttgatcacactattttagagtcttaatgccaagtcagcagatttgctttatgaattacagggactagaaatgcccacattcaggaaatttgtaataacattgtctagacacctatcctcattctagtagaaagtgtgtacatactgtaaatatgtgtgattgcttgacttgaaaaggtttgaattctgaatgttataccatccttgtaagtaagtttgtaatttccaccataaattatggtaaatataaaactccagaggttgttctactccatacagttcacactgattgtgacacattcttagtagctagtgtctgttctagtcactgcactggagtctacgagccggaactcgctatatgcacgtgtgtgtgtccgtatgtaagaaagtgtgcaccgagtgactgaatggttgagatgaattggaatgctgaagactaacgaagaaactagagactgatatcgagcattctgcccacctcgctctgtatttaattaattgtgctatatgttgctttaacaacccattgagcagtcagggaatgtgagtaagcttgctgccaaaggtaactaggaaagcattcatctgctgcctccttgtttttgctcctagagagtgaaaatacaggcaattttactgtgagtgtttcactggaaatgtacaatctttgtgtgttagagtatttgttttagtaagaaatgtttacacagcttgtggaattatttcgtgggaaaataaatttttataacttctcccaaaaaaaaaaaaaaaaa

SEQ ID NO: 8 - DNA, 3322 bp, Homo sapiens
gaggaggaggggacccgccgccgggggctggctgcttcgctccgagccgacttttcgccaatggtccaaagggacatgtccaagtcgcctcccacagcggcggcggcggtggcgcaggagatccagatggaactgctagagaacgtggctcccgcgggggcgctcggagccgccgcacagtcatatggaaaaggagccagaaggaaaaacagatttaaaggatctgatggaagcacgtcatctgatactacctcaaatagttttgttcgccagggttcggcagactcctacactagccgtccatccgattccgatgtatctctggaggaggaccgggaggcagtgcgcagagaagcggagcggcaggcccaggcacagttggaaaaagcaaagacaaagcccgttgcatttgcggttcggacaaatgtcagctacagtgcggcccatgaagatgatgttccagtgcctggcatggccatctcattcgaagcaaaagattttctgcatgttaaggaaaaatttaacaatgactggtggatagggcgattggtaaaagaaggctgtgaaatcggattcattccaagcccagtcaaactagaaaacatgaggctgcagcatgaacagagagccaagcaagggaaattctactccagtaaatcaggaggaaattcatcatccagtttgggtgacatagtacctagttccagaaaatcaacacctccatcatctgctaagcagaagcagaagcacactcctccgtatgatgtggtaccttccatgcgaccagtggtcctagtgggcccttctctgaagggctacgaggtcacagatatgatgcaaaaagcgctgtttgattttttaaaacacagatttgaagggcggatatccatcacaagggtcaccgctgacatctcgcttgccaaacgctcggtattaaacaatcccagtaagcacgcaataatagaaagatccaacacaaggtcaagcttagcggaagttcagagtgaaatcgaaaggatttttgaacttgcaagaacattgcagttggtggtccttgacgcggatacaattaatcatccagctcaactcagtaaaacctccttggcccctattatagtatatgtaaagatttcttctcctaaggttttacaaaggttaataaaatctcgagggaaatctcaagctaaacacctcaacgtccagatggtagcagctgataaactggctcagtgtcctccagagctgttcgatgtgatcttggatgagaaccagcttgaggatgcctgtgagcaccttgccgactatctggaggcctactggaaggccacccatcctcccagcagtagcctccccaaccctctccttagccgtacattagccacttcaagtctgcctcttagccccaccctagcctctaattcacagggttctcaaggtgatcagaggactgatcgctccgctcctatccgttctgcttcccaagctgaagaagaacctagtgtggaaccagtcaagaaatcccagcaccgctcttcctcctcagccccacaccacaaccatcgcagtgggacaagtcgcggcctctccaggcaagagacatttgactcggaaacccaggagagtcgagactctgcctacgtagagccaaaggaagattattcccatgaccacgtggaccactatgcctcacaccgtgaccacaaccacagagacgagacccacgggagcagtgaccacagacacagggagtcccggcaccgttcccgggacgtggatcgagagcaggaccacaacgagtgcaacaagcagcgcagccgtcataaatccaaggatcgctactgtgaaaaggatggagaagtgatatcaaaaaaacggaatgaggctggggagtggaacagggatgtttacatcccccaatgagttttgcccttttgtgtttttttttttttttttttgaagtcttgtataactaacagcatccccaaaacaaagtctttggggtctacactgcaatcatatgtgatctgtcttgtaatattttgtattattgctgttgcttgaatagcaatagcatggatagagtattgagatactttttcttttgtaagtgctacataaattggcctggtatggctgcagtcctccggttgcatactggactcttcaaaaactgttttgggtagctgccacttgaacaaaatctgttgccacccaggtgatgttagtgttttaagaaatgtagttgatgtatccaacaagccagaatcagcacagataaaaagtggaatttcttgtttctccagatttttaatacgttaatacgcaggcatctgatttgcatattcattcatggaccactgtttcttgcttgtacctctggctgactaaatttggggacagattcagtcttgccttacacaaaggggatcataaagttagaatctattttctatgtactagtactgtgtactgtatagacagtttgtaaatgttatttctgcaaacaaacacctccttattatatataatatatatatatatatcagtttgatcacactattttagagtcttaatgccaagtcagcagatttgctttatgaattacagggactagaaatgcccacattcaggaaatttgtaataacattgtctagacacctatcctcattctagtagaaagtgtgtacatactgtaaatatgtgtgattgcttgacttgaaaaggtttgaattctgaatgttataccatccttgtaagtaagtttgtaatttccaccataaattatggtaaatataaaactccagaggttgttctactccatacagttcacactgattgtgacacattcttagtagctagtgtctgttctagtcactgcactggagtctacgagccggaactcgctatatgcacgtgtgtgtgtccgtatgtaagaaagtgtgcaccgagtgactgaatggttgagatgaattggaatgctgaagactaacgaagaaactagagactgatatcgagcattctgcccacctcgctctgtatttaattaattgtgctatatgttgctttaacaacccattgagcagtcagggaatgtgagtaagcttgctgccaaaggtaactaggaaagcattcatctgctgcctccttgtttttgctcctagagagtgaaaatacaggcaattttactgtgagtgtttcactggaaatgtacaatctttgtgtgttagagtatttgttttagtaagaaatgtttacacagcttgtggaattatttcgtgggaaaataaatttttataacttctcccaaaaaaaaaaaaaaaaa

SEQ ID NO: 9 - DNA, 3927 bp, Homo sapiens
tttttgtaacttacttgttttagtatatactgatctgaacaggcaaaaccccatgttagcaacagctcggtcaggaagagaaatgctaccttttctagatgtttgccgggaggcagccccctgaaaggagatctcaaattacctgtaagaaaaaaaatttttttaaaggcagtcagagagagcaaagttttggcatgcctgcaaaaacagcagtgtctgtaaaaactacctagagacagcagctaagtgttgatttgcccatgactcttacctgggagtggaaggtgggaagaaatggacagtggccgtgacgagaagctgaggcacggtgcagcttggtgaagccacgctctgactgcgtcctgccacctcttcatgcagtgctgcgggctggtacatcgccggcgagtacgggtgtcctatggttcggcagactcctacaccagccgcccatccgattcagatgtgtctttggaagaggaccgggaggcagtccgtagagaagctgagcggcaggcccaggcccagttggaaaaagcaaagacaaaacctgttgcatttgcagttcggaccaatgtcaggtacagcgcagcccaggaggatgacgtcccggtgcccggcatggccatctcattcgaggcaaaagattttctgcatgttaaggaaaaatttaataatgactggtggataggacggctggttaaagaaggctgtgaaatcggatttattccaagcccggtcaaactagagaacatgaggctacagcatgaacagagagccaagcaagggaaattctactccagtaaatcaggaggaaattcatcatccagtttgggtgacatagtacccagttccagaaaatcgacacctccatcatctgctatagacatagatgctactggcttagatgcagaagagaatgatattccagcaaaccaccgctcccctaagcccagtgcaaacagtgtaacgtcaccccactccaaagagaaaagaatgcccttctttaagaagacagagcacactcctccttacgatgtggtaccatccatgagaccagtggtgttggtgggcccctccctgaaggggtatgaggtcacagatatgatgcaaaaagcactgtttgattttctaaaacacagatttgaaggacggatatccatcacaagagtcactgctgacatctccctggccaaacgctcagtattaaacaaccccagtaagcacgcaataatagaaagatccaacacaaggtcgagcttagcggaagttcagagtgaaattgaaaggatttttgaacttgcaagaacactgcaattggtagtccttgacgcggatacaattaatcacccagctcaactcagtaagacctctctggcccctatcatagtgtatgtaaagatttcttctcctaaggttttacaaaggttaataaaatctcgaggaaagtctcaagcaaagcacctcaatgtccagatggtggcagccgataaactggcccagtgtcccccgcaggagtcatttgatgtgatcttggatgagaaccaacttgaggatgcttgtgagcatctcgccgactatctggaggcgtactggaaggccacccaccctcccagcagtaacctccccaaccctctccttagccggactttagccacctcaactttacctctgagccccacccttgcctctaattcacagggttctcaaggtgatcagaggactgatcgctctgctccccggtctgcttcccaagctgaagaagaaccttgtctggaaccagtcaaaaaatcccaacaccgttcttcctcagccacacaccaaaaccaccgcagtgggacaggtcgaggcctctctaggcaagaaacgtttgactctgaaacccaagagagccgagactctgcctacgtggagccaaaggaagattattcacatgaacatgtggaccgctatgttccacaccgtgaacataaccacagagaggagagccacagcagcaatggccacaggcacagggagcctcgccaccgcactagggacatgggtcgagaccaagaccacaatgagtgcagcaaacaacgaagccggcataaatctaaggatcgctactgtgacaaggaaggggaagtaatatccaaaagaaggagtgaggctggcgagtggaacagggatgtatacatccgccaatgaccgtgcgtgtctctgcccccaagtcttgtgtaacatcatccttaagcaaaatctttggggctccattgcaaccatctgtgatctgtcttgtatattttgtattgctgttgcttgaatagcaatagcatgcaatcaagtattactatacattcttttgtaagtaccacagaaattggcctagtacaagctgcagtcctcagtctatatcctgaactcttcaaaagctgtttggggtagctgccacctgaacagagtggcttcagaaatatagttaatgtattcagtgagccagatgcagcacagataacaagtggaattcctccatatttttaatacccagatacctgacctgtcattctttcatggaccactgtttcttgcttgtacctctggctgactaaatttggggacagattcagtcttgccttacacaaaggggattataaagttagaatctattttctatgtactagtactgtgtactgtgtagacagtttgtaaatgttatttctgcaaacaccttattataattatatatataatatatatatatatatcagtttgatcacacaattttagggtcttaatgccaagtcagcagatttgctttatgaatcacagggactagaaatgcccacattcaggaaatttctaatattgtctagactcctctccctattctaagagagagcctgtatatagtgtaaatacgtgtgattgcttgacttgaggtttgatttctgaatgtaatcccatccttgtaagtttttcattttaaccataaattatggtaaatgtaaccaactccagaggtttttctactccatacagtccacactgattgtgacaaacacgttcttagcagctagtgtctgtggtagtcactgcactggagtatccaagcggagcccaccatgtgcacatgcgtgtgtgagtgtgagagtgagtggctgagacgaagtggaatgcagaagactaatgcggaaaccagaggctgatggacctggctttgcatttaactgttgtatgttgctttcgaacccactgagcagccagggaatgtgaggaagcttgctgccaaaggaactagggaagcacttagtggctgactccctgcttttgcccctgaagagagaaaacatgggcgtcttgtactgtgagtgttccactggaaatggacaatctttgtgtgtcagagtattttgttttagtaagaaatgtttacacagcttgtgcaattatttcgtagggaaataaatttttataacttgtcccacttcactttctaaacgtgcctattgctcctcttgttcatcctcagccaccgctcttcttcccctgcaccaagctgtgcagctgcactgtcggaatctagaagtgcttcttagaaccaaagttcctgctaaatctttagaagttatcagggcaaaaatggaatgacttgaagcaaaaaaagttgaaacattttcttttacgagacatctctaagggagaacgtagaagacacttttttcacacctcaatttgctttcgttaagaagcttaaactgctcttagccgaggctatccaatctctgtctctcaagagcccagtgctgatggagaggtaagactgtatgagctccacacgcccctagcattcgaagccttcgcaggaaaatgtctatctgtgattacccaaccagatcagtatatactaaaacaagtaagttacaataaatatatttttgttttgtt

SEQ ID NO: 10 - DNA, 21 bp, Homo sapiens
aacatgaggctacagcatgaa